(19) United States
(12) Patent Application Publication
(10) Pub. No.: US 2026/0102771 A1
(43) Pub. Date: Apr. 16, 2026

(54) GENOME-WIDE qPCR

(71) Applicant: The Trustees of Columbia University in the City of New York, New York, NY (US)

(72) Inventors: Zhilun Zhao, New York, NY (US); Naixin Qian, New York, NY (US); Wei Min, New York, NY (US)

(21) Appl. No.: 19/361,996

(22) Filed: Oct. 17, 2025

Related U.S. Application Data
(63) Continuation of application No. PCT/US2024/025082, filed on Apr. 18, 2024.
(60) Provisional application No. 63/460,693, filed on Apr. 20, 2023.
(60) Provisional application No. 63/463,357, filed on May 2, 2023.

(51) Int. Cl. B01L 3/00; C12Q 1/686

(52) U.S. Cl. CPC B01L 3/502761; B01L 3/502707; C12Q 1/686; B01L 2200/0647; B01L 2200/12; B01L 2200/16; B01L 2300/0819; B01L 2300/0893; B01L 2300/12

(57) ABSTRACT

A massively multiplexed qPCR technique that allows genome-wide qPCR (100-100,000 targets simultaneously) with few-hour turnaround and at lost cost. A massively multiplexed isothermal amplification technique that allows genome-wide multiplexed isothermal amplification (100-100,000 targets simultaneously) with few-hour turnaround and at lost cost. Microwell array chips, isothermal amplification imaging devices and qPCR imaging devices for use with the techniques are provided also.

### CROSS-REFERENCE TO RELATED APPLICATIONS

This application is a continuation of PCT International Application No. PCT/US2024/025082, filed Apr. 18, 2024, which claims the benefit of U.S. Provisional Application Nos. 63/463,357, filed May 2, 2023, and 63/460,693, filed Apr. 20, 2023, the contents of each of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### STATEMENT REGARDING FEDERALLY SPONSORED RESEARCH OR DEVELOPMENT

This invention was made with government support under EB029523 awarded by the National Institutes of Health. The government has certain rights in the invention.

### BACKGROUND OF THE INVENTION

Identifying nucleic acids and measuring their concentrations are among the core methodologies in modern bioscience. For example, in diagnostics, nucleic acids are commonly used to identify pathogens; in basic research, mRNA concentrations are measured to investigate biological responses to stimuli. For relatively low multiplexity (i.e., one or a small number of targets of interest to be measured), quantitative polymerase chain reaction (qPCR) is widely used for its simple workflow, quick turnaround, and low cost. On the other hand, next-generation sequencing (NGS) is used if a comprehensive understanding of the entire transcriptome is needed, at the expense of a complex workflow, slow turnaround, and high cost.

### BRIEF SUMMARY OF THE INVENTION

A massively multiplexed qPCR technique that allows genome-wide qPCR (e.g., about 100-100,000 targets simultaneously) with a few-hour turnaround and at low cost is described herein. An isothermal amplification technique for multiplexing with a quick turnaround and low cost is also described herein. Related microwell array chips, systems, devices, and methods of their manufacture are also described herein.

According to certain embodiments of the present disclosure, a microwell array chip is provided comprising a plurality of microwells,
wherein each microwell of the plurality has a microbead contained therein which has, covalently or non-covalently attached thereto, at least one pair of primers comprising a forward and reverse primer specific for a predefined nucleic acid, and wherein
(a) each microbead comprises one, or more, types of Raman-active small molecule(s) (RASMs), each at a predefined concentration in the microbead, or one or more, types of infrared-active (IR) small molecule(s) (IRASMs), each at a predefined concentration in the microbead, and
(b) the Raman or IR spectra of each microbead containing the RASMs or IRASMs is individually resolvable from that of every other microbead in the plurality of microwells having a different at least one pair of primers comprising a forward and reverse primer specific for a predefined nucleic acid.

Also provided is a system comprising (i) a detector component which can detect a light signal from a marker within one or more microwells of a microwell array chip, which marker changes its detectable signal upon binding to double-stranded DNA, and (ii) a microwell array chip as described herein.

Also provided is a method for determining the presence of one or more predefined nucleic acids in a sample, the method comprising adding the sample to (a) a microwell of a microwell array (MWA) chip described herein or (b) to a microwell of a microwell array (MWA) chip of a system described herein, wherein the MWA chip contains a microwell for each predefined nucleic acid having a microbead therein with a pair of primers covalently or non-covalently attached thereto comprising a forward and reverse primer specific for said predefined nucleic acid, and cleaving the covalent or non-covalent bond between each primer and the microbead and having therein provided, or adding, reagents sufficient to permit a polymerase chain reaction to occur therein, so as to produce one or more double-stranded amplicons of the predefined nucleic acid(s), and detecting the presence of the double-stranded amplicons, wherein presence of the double stranded amplicons indicates that the predefined nucleic acid is/are present in the biological sample.

Also provided is a method for determining the presence of one or more predefined nucleic acids in a sample, the method comprising adding the sample to (a) a microwell of a microwell array (MWA) chip described herein or (b) to a microwell of a microwell array (MWA) chip of a system described herein, wherein the MWA chip contains a microwell for each predefined nucleic acid having a microbead therein with at least one pair of primers covalently or non-covalently attached thereto comprising a forward and reverse primer specific for said predefined nucleic acid, and cleaving the covalent or non-covalent bond between each primer and the microbead and having therein provided, or adding, reagents sufficient to permit an isothermal amplification reaction to occur therein, so as to produce one or more double-stranded amplicons of the predefined nucleic acid(s), and detecting the presence of the double-stranded amplicons, wherein presence of the double stranded amplicons indicates that the predefined nucleic acid is/are present in the biological sample.

A multi-well plate comprising a plurality of wells, wherein each well is not in fluid contact with any adjacent well and has a volume of between 0.5 milliliters and 5 milliliters, and wherein each well further comprises, on a bottom surface thereof, a microwell array chip described herein.

A method of manufacturing a microwell array chip comprising
printing a microwell array design on a soda lime photomask;
spin-coating a photoresist layer onto a silicon wafer;
exposing through the photomask, developing and hard-curing the photoresist layer on the silicon wafer so as to produce a microwell array-patterned silicon wafer;
vacuum-depositing trichloro(1H, 1H,2H,2H-perfluorooctyl) silane onto the silicon wafer;
pouring uncured and degassed PDMS polymer onto the patterned silicon wafer; and a) heat curing the PDMS polymer so as to form a PDMS microwell array chip; or b) placing a thin coverglass on top so that the PDMS polymer is sandwiched between the patterned silicon wafer and coverglass and applying pressure and heat to spread the PDMS across the coverglass substantially evenly and pushing the patterned silicon wafer to touch the coverglass, while applying heat for rapid curing so as to form a glass-bottomed PDMS microwell array chip; and
removing the PDMS or glass-bottomed PDMS microwell array chip from the patterned silicon wafer; and
adding to each microwell of the microwell array chip a predetermined microbead comprising one, or more, types of Raman-active small molecule(s) (RASMs), each at a predefined concentration in the microbead, or one or more, types of infrared-active (IR) small molecule(s) (IRASMs), each at a predefined concentration in the microbead,
thereby producing a microwell array chip.

A method is provided for determining the presence of one or more predefined nucleic acids in a sample, the method comprising adding the sample to a microwell of the (a) microwell array (MWA) chip as described herein or (b) microwell array (MWA) chip of the system as described herein, wherein the MWA chip contains a microwell for each predefined nucleic acid having a microbead therein with a pair of primers comprising a forward and reverse primer specific for said predefined nucleic acid, and cleaving the covalent or non-covalent bond between each primer and the microbead and having therein provided, or adding, reagents sufficient to permit a polymerase chain reaction to occur or reagents sufficient to permit an isothermal amplification reaction to occur therein, so as to produce one or more double-stranded amplicons of the predefined nucleic acid(s), and isolating the double-stranded amplicons from each microwell and performing a sequencing technique thereupon.

A method is provided for determining the presence of one or more predefined nucleic acids in a sample, the method comprising adding the sample to a microwell of the (a) microwell array (MWA) chip comprising a plurality of microwells,
wherein each microwell of the plurality has a microbead contained therein which has, covalently or non-covalently attached thereto, at least one pair of primers comprising a forward and reverse primer specific for a predefined nucleic acid,
and wherein the microbead of each microwell is individually resolvable from that of every other microbead in the plurality of microwells having a different at least one pair of primers comprising a forward and reverse primer specific for a predefined nucleic acid, wherein the MWA chip contains a microwell for each predefined nucleic acid having a microbead therein with a pair of primers comprising a forward and reverse primer specific for said predefined nucleic acid,
and cleaving the covalent or non-covalent bond between each primer and the microbead and having therein provided, or adding, reagents sufficient to permit a polymerase chain reaction to occur therein, or reagents sufficient to permit an isothermal amplification reaction to occur therein, so as to produce one or more double-stranded amplicons of the predefined nucleic acid(s).

### BRIEF DESCRIPTION OF THE DRAWINGS

FIGS. 1A-1B: FIG. 1A) An embodiment of a chip design including magnified detail of a non-limiting microwell design (top view); FIG. 1B) Three panels showing other microwell designs from the top view perspective. The far right-hand panel shows microwell design with no spatially separated signal detection portion. This latter design can be used, for example, where the signal is measured from above or below in line of the microbead.

FIG. 2 shows an example of RASMs beads barcoded with brightness level and differences in spectra so as to be individually resolvable. In comparison, most fluorescence-based techniques only have about three spectrally resolvable fluorescence dyes usable at the same time, which greatly limits multiplexity.

FIG. 3 shows, at the top, a stylized forward primer and reverse primer (i.e., a primer pair) each attached via a poly(U) linker to a microbead (which itself is brightness and spectra barcoded as described herein); beneath the microbead image is a stylized microwell array (MWA) chip with different barcoded microbeads retained in different individual microwells. The spatial location or coordinates of each bead type in the MWA (and therefore each specific primer pair or target nucleic acid sequence) can be recorded before use of the methods described herein, after addition of the sample, or after a PCR reaction has completed.

FIG. 4: An embodiment of a detection method where the poly(U) type primer-microbead attachment approach is used, and a sample is placed in microwells along with PCR reagents, a double-stranded DNA detection molecule, and a USER enzyme which cleaves the forward and reverse primers from their microbead by breaking the poly(U) linker. After subjecting the contents of the microwells to PCR conditions, the amplicons produced (if any) are detected in a given microwell, which is known (e.g., through spatial coordinates and/or microbead type) or is then determined to correspond to a particular predefined target nucleic acid sequence. Presence and/or quantitative assessment of one or more predefined target nucleic acid sequences in the sample is simultaneously provided.

FIG. 5: Non-limiting examples of IRASMs that can be used in certain embodiments. A spectral range of ˜200 cm⁻¹ can be covered with thiocyanate and nitrile molecules.

FIG. 6: Non-limiting examples of IRASMs that can be used in certain embodiments, including a microbead loading option. Examples of several RASMs are also listed.

FIG. 7: Non-limiting examples of IRASMs that can be used in certain embodiments.

FIG. 8: Non-limiting examples of IRASMs that can be used in certain embodiments.

FIG. 9: Non-limiting examples of IRASMs that can be used in certain embodiments. See Anderson, B. A., Bell, E. C., Ginah, F. O., Harn, N. K., Pagh, L. M., & Wepsiec, J. P. (1998) “Cooperative catalyst effects in palladium-mediated cyanation reactions of aryl halides and triflates.” The Journal of Organic Chemistry, 63 (23), 8224-8228. See also Yang, C. & Williams, J. M. (2004) “Palladium-catalyzed cyanation of aryl bromides promoted by low-level organotin compounds.” Organic Letters, 6(17), 2837-2840.

FIG. 10: Non-limiting examples of IRASMs that can be used in certain embodiments.

FIG. 11: Non-limiting examples of IRASMs that can be used in certain embodiments.

FIG. 12: Non-limiting examples of IRASMs that can be used in certain embodiments.

FIG. 13: Non-limiting examples of IRASMs that can be used in certain embodiments.

FIGS. 14A-14C: FIG. 14A shows a sample image of the mixed VibrantBeads, which were pseudo colored according to their Raman spectra. FIG. 14B shows a representative view of spectral and intensity barcoding of VibrantBeads from FIG. 14A. Three channels (Ch1, Ch2, and Ch3) were shown to spectrally barcode the beads. At the same time, there were 8 intensity levels (lv8, lv7, etc.) for each channel to intensity barcode the beads. FIG. 14C shows cluster analysis of a total of 128 kinds of barcoded VibrantBeads shown with 3 channels from two combinations of 3 dyes (e.g., dye1+dye shown as Channel 1 and Channel 2; and dye 1+dye 3 shown as Channel 1 and Channel 3). Each cluster shows one barcode where a total of 128 clusters are shown for 128 kinds of barcodes. In total, 5376 kinds of VibrantBeads were made by adopting intensity barcoding, spectral barcoding, and size barcoding.

FIG. 15: The schematic shows the principal design of the micro-PCR array chip. Upper left: A schematic showing the designed feature of the micro-PCR array chip. Each unit contains a VibrantBeads housing and a connected PCR chamber. Units are connected to manifolds for sample loading. Vacuum batteries are alongside the units to assist sample flow-through and suppress bubble forming. Color-coded VibrantBeads were placed in the microwell, whose identities were decoded by reading out the color-codes (shown in upper right insert.). In total, there are 10,000 units on a chip. Lower left: A protype chip is shown side-by-side with a quarter coin to indicate the scale. Holes were drilled to load the sample. After the chips were made, they were placed in a vacuum chamber to charge the vacuum battery. Lower right: A zoom-in view of the chip. The narrow channels are the manifolds connecting the sample inlets to the microwells. The wide channels alongside the microwells are vacuum batteries that have dead ends. Once the chips are placed in the vacuum chamber, the residue air will escape from the chip as PDMS is gas permeable and will leave the space vacuum. When the sample is added, since the vacuum batteries are not connected to manifolds, they will remain in vacuum and serve as a low-pressure dump to create pressure gradients to help the sample liquid flow into the microwells. Upper right: loaded VibrantBeads. VibrantBeads are loaded into the bead housing next to the PCR chambers. The pseudo color indicates color-codes which indicate the identity of the beads.

FIG. 16: The image shows on-chip PCR detection of COVID-19 micro-PCR array chips fabricated as described above with VibrantBeads for COVID detection added. Then samples with and without COVID DNA present were added to separate chips and a PCR reaction was performed. At the end of the reaction, fluorescence images were acquired. The left panel shows end results of the negative control where no fluorescence signal from SYBR Green was detected, whereas the right panel shows positive fluorescence results when COVID-19 reverse-transcribed DNA was present. The SYBR Green signal was normalized to loading control in the sample for head-to-head comparison. Scale bars: 500 μm.

FIG. 17: The figure illustrates the schematics of using IR active molecules to color-code beads.

FIG. 18: This figure shows the calculation results of various nitrile moieties that have resolvable IR spectra for the potential color coding.

FIG. 19: This figure shows preliminary data to demonstrate the feasibility of using IR active molecules for color-coding the beads. Here, the polymer monomers were modified to contain nitrile groups on their side chains, and the monomer solution was made into droplets through microfluidic devices. The droplets were then placed under UV illumination to initiate polymerization to form hydrogels. As a result, the IR active nitrile groups were covalently attached to the polymer backbone stoichiometrically. The graph on the bottom indicates a signature IR peak from the nitrile group.

FIG. 20: This figure shows the preliminary results of using hydrogel beads as carriers of PCR primers. Hydrogel beads made by the method described in the previous figure were loaded into microwells dried. Then samples with and without COVID DNA present were added to separate chips and a PCR reaction was performed. At the end of the reaction, fluorescence images were acquired. The left panel shows end results of the negative control where no fluorescence signal from SYBR Green was detected, whereas the right panel shows positive fluorescence results when COVID-19 reverse-transcribed DNA was present. The SYBR Green signal was normalized to loading control in the sample for head-to-head comparison. Scale bars: 100 μm.

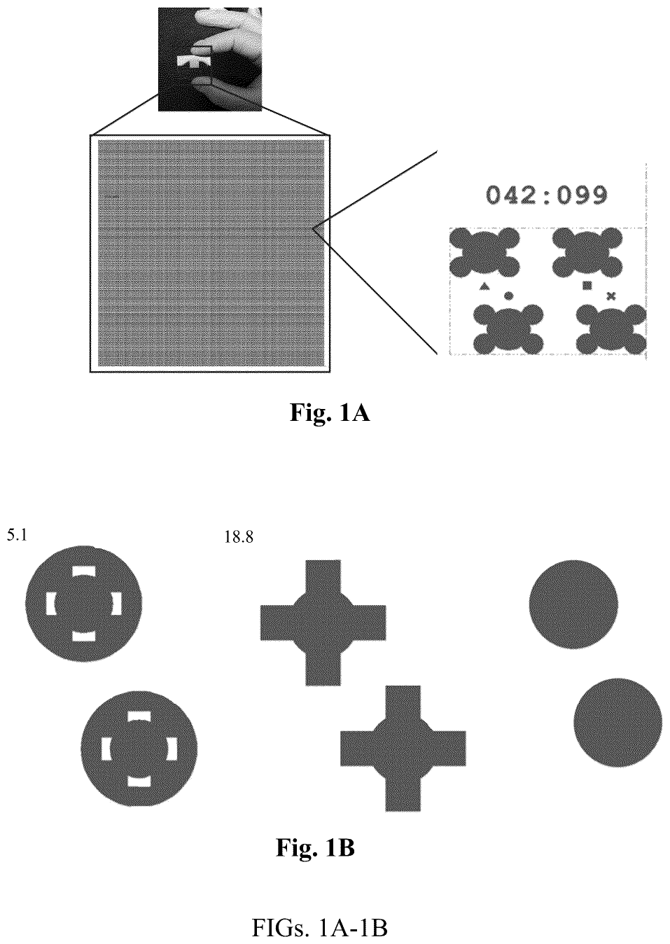

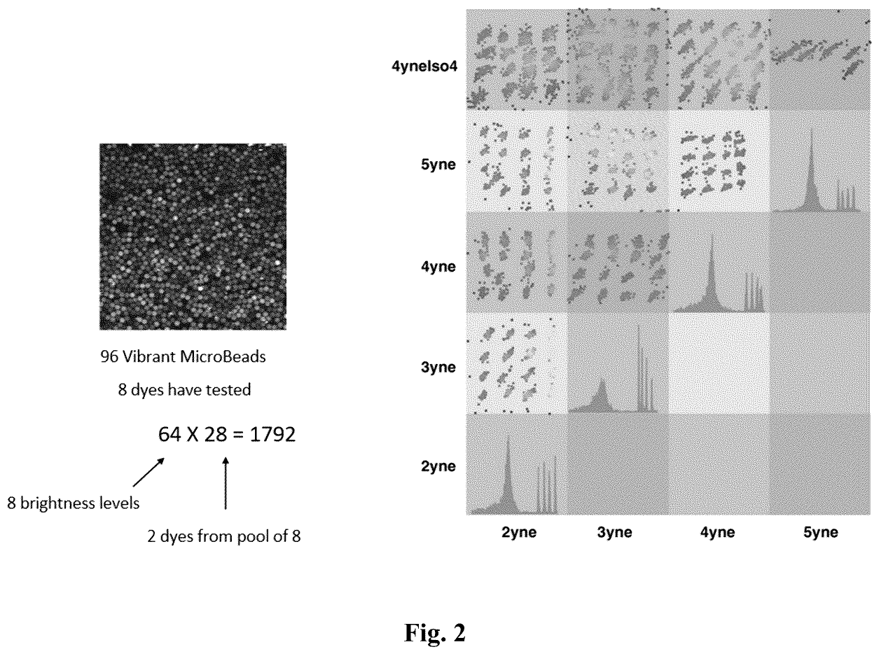

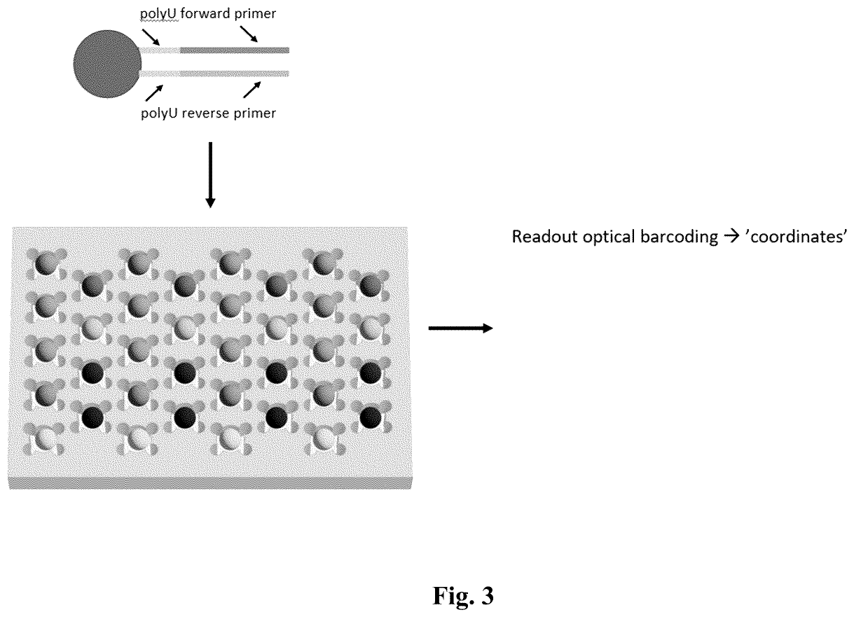

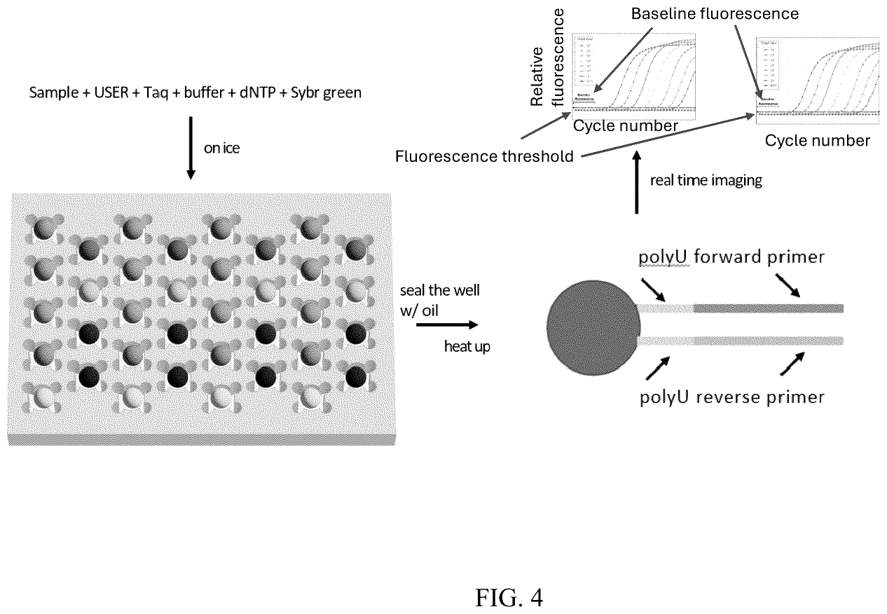

### DETAILED DESCRIPTION OF THE INVENTION

A massively multiplexed qPCR technique that allows genome-wide qPCR (100-100,000 targets simultaneously) with a few-hour turnaround and at a lost cost is described herein. This technology bridges the gap between current qPCR and NGS techniques, and will allow rapid on-the-spot determination of, for example, viral or bacterial infections in human patient samples. qPCR imaging devices for use with the technique are also provided, including handheld versions, as well as microwell array chips that can be used with the technique. A massively multiplexed isothermal amplification technique that can act on 100-100,000 targets simultaneously is also described, as well as isothermal amplification imaging devices for use with the technique.

In some embodiments, a microwell array chip is provided comprising a plurality of microwells,
wherein each microwell of the plurality has a microbead contained therein which has, covalently or non-covalently attached thereto, at least one pair of primers comprising a forward and reverse primer specific for a predefined nucleic acid, and wherein (a) each microbead comprises one, or more, types of Raman-active small molecule(s) (RASMs), each at a predefined concentration in the microbead, or one or more, types of infrared-active (IR) small molecule(s) (IRASMs), each at a predefined concentration in the microbead, and (b) the Raman or IR spectra of each microbead containing the RASMs or IRASMs is individually resolvable from that of every other microbead in the plurality of microwells having a different at least one pair of primers comprising a forward and reverse primer specific for a predefined nucleic acid.

In embodiments, a microwell is provided comprising a microbead contained therein which has, covalently or non-covalently attached thereto, at least one pair of primers comprising a forward and reverse primer specific for a predefined nucleic acid, and wherein (a) the microbead comprises one, or more, types of Raman-active small molecule(s) (RASMs), each at a predefined concentration in the microbead, or one or more, types of infrared-active (IR) small molecule(s) (IRASMs), each at a predefined concentration in the microbead.

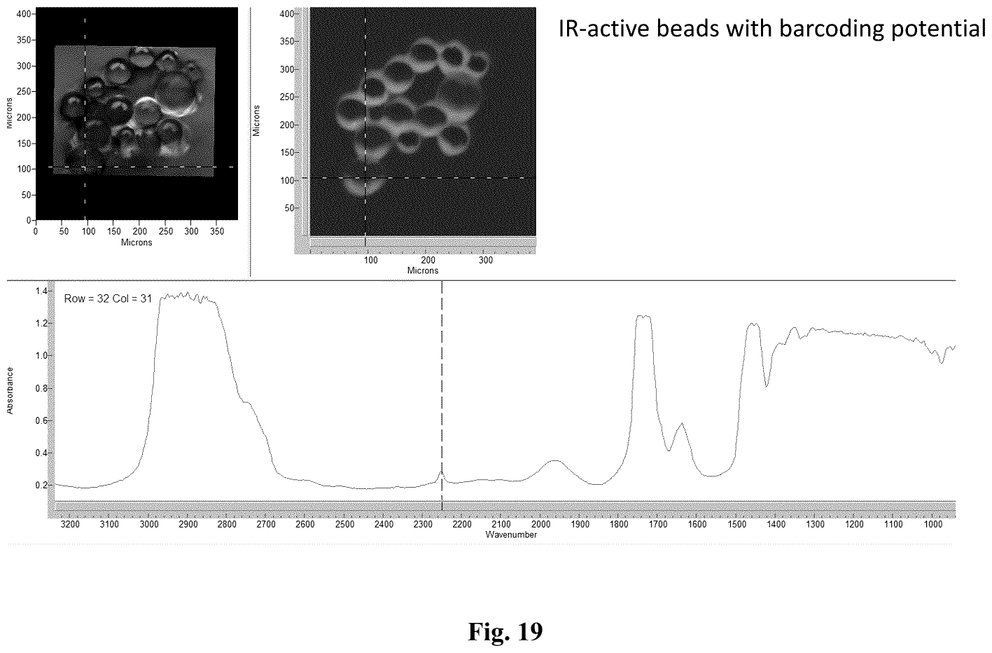

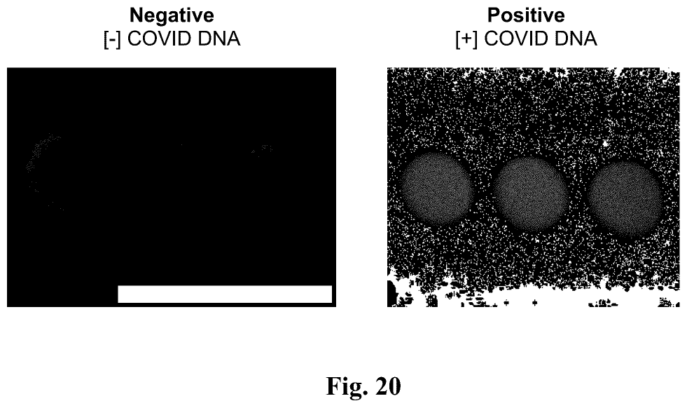

In some embodiments, a microwell is provided comprising an IR color-coded microbead. In addition to the non-covalent absorption of the dyes into the beads, these functional groups can be covalently linked to the matrix of the beads. As a non-limiting example, FIG. 19 shows where nitrile groups were used as the side-chain of the monomer. After in situ polymerization to form beads, these nitrile groups were then covalently incorporated into the beads.

In embodiments, each microwell of the plurality is not in fluid contact with any adjacent microwell.

In embodiments, the plurality of microwells comprises 100,000 or more microwells.

In embodiments, the plurality of microwells comprises 500,000 or more microwells.

In embodiments, each microwell of the plurality is sized such that only one microbead can be contained therein. In some embodiments, each microwell of the plurality comprises only one microbead.

In embodiments, each microwell of the plurality comprises a polydimethylsiloxane wall and bottom, or comprises a polydimethylsiloxane wall and a glass bottom.

In embodiments, surfaces of the microwell, which surfaces are in contact with a fluid sample when a fluid sample is placed in the microwell, are functionalized with amine groups.

In embodiments, the amine-functionalized surfaces increase hydrophilicity so as to increase interaction between well walls and microspheres or microbeads as compared to a non-functionalized surface of the microwell.

In embodiments, each microbead comprises a polystyrene particle. In embodiments, the beads are paramagnetic or magnetic microbeads.

In embodiments, each microbead has an average diameter of single or double-digit microns. In embodiments, each microbead has an average diameter of at least 0.5 μm. In embodiments, each microbead has an average diameter of at least 1.0 μm. In embodiments, each microbead has an average diameter of at 0.5 μm. In embodiments, each microbead has an average diameter of 1.0 μm.

In embodiments, each microbead, in an embodiment where RASMs are used, has peak Raman shift at a predetermined stimulation wavelength of at least 10 cm⁻¹ less or 10 cm⁻¹ more than the peak Raman shift of all the other bead types.

In embodiments, the RASMs are alkyne-containing and do not exceed a molecular weight of 350 g/mol.

In embodiments, the Raman-active small molecules comprise one or more of the following:

In embodiments, the Raman-active small molecules comprise one or more of the following:

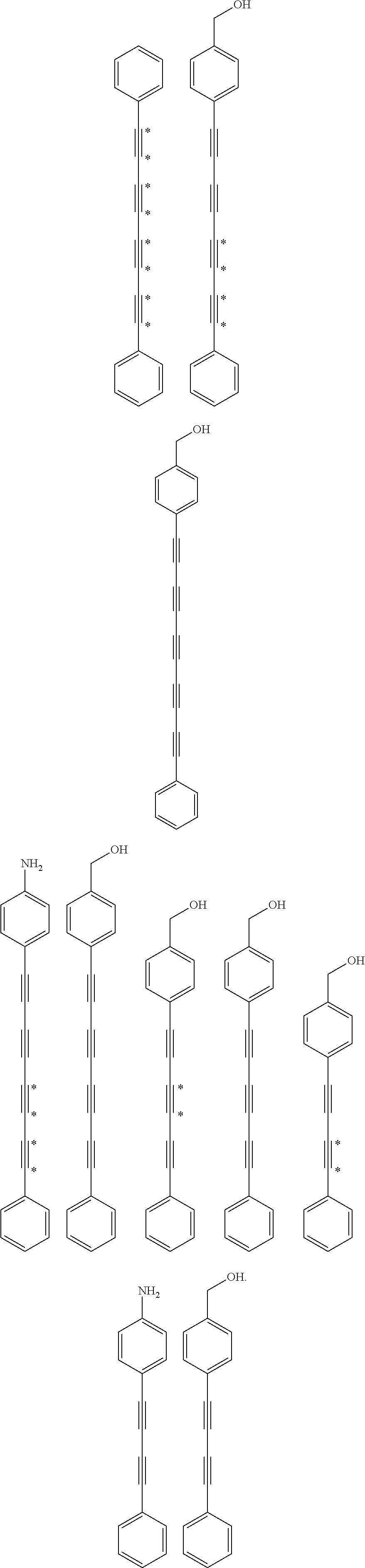

In embodiments, the Raman-active small molecules comprise one or more of the following, wherein “*” adjacent to an alkyne carbon atom indicates presence of a ¹³C isotope:

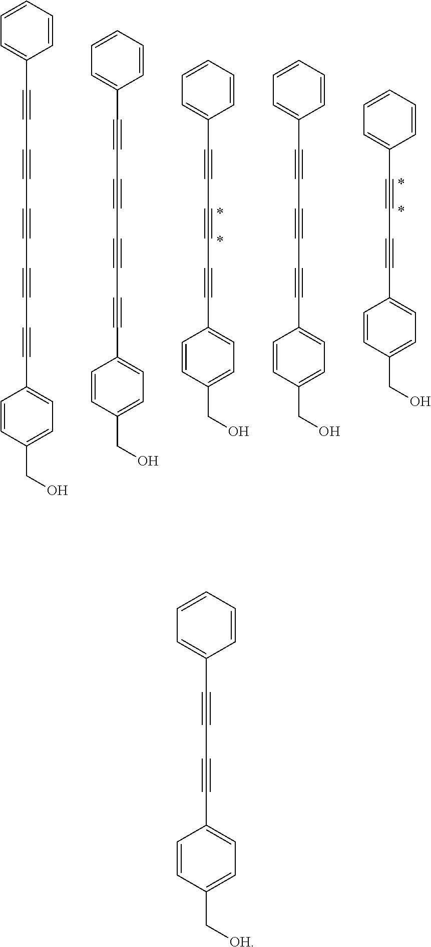

In embodiments, the identity of a microbead in a microwell is determined with Stimulated Raman Scattering (SRS) or Spontaneous Raman Scattering. In embodiments, the “identity” of a microbead is seen by knowing which predefined nucleic acid the microbead has primers initially attached specific therefor. can be recorded before use in the methods herein or after addition of the sample, or after the PCR reaction has completed he identity can be can be recorded or determined before use in the methods herein, or after addition of the sample, or after the PCR reaction has completed.

In embodiments, wherein RASMs are used, the SRS can be performed using a 532 nm laser.

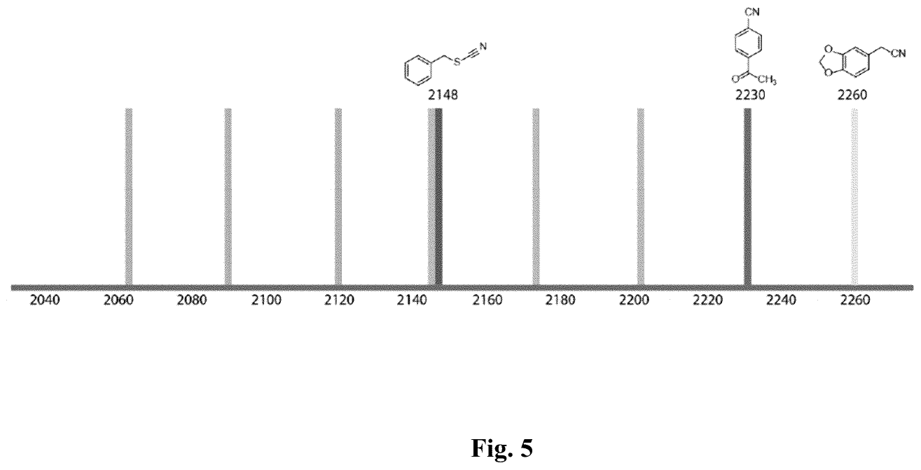

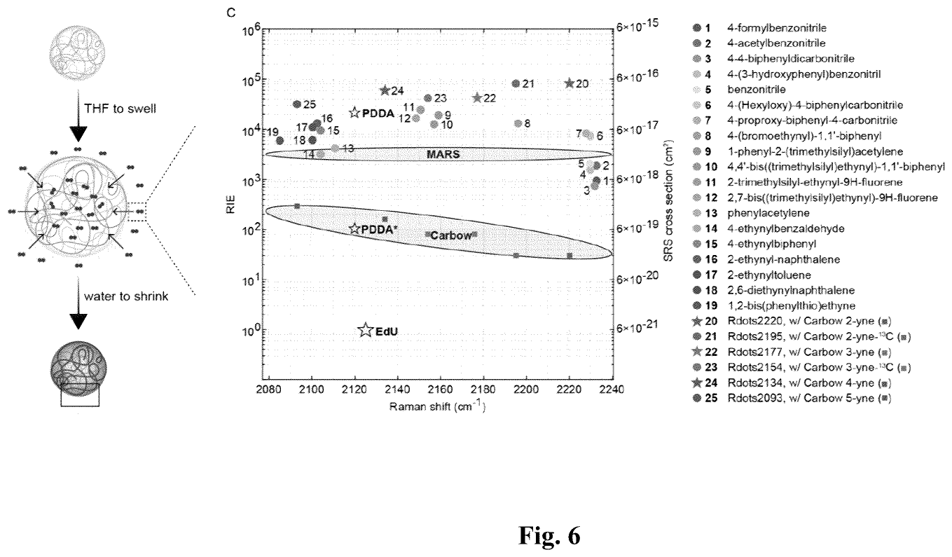

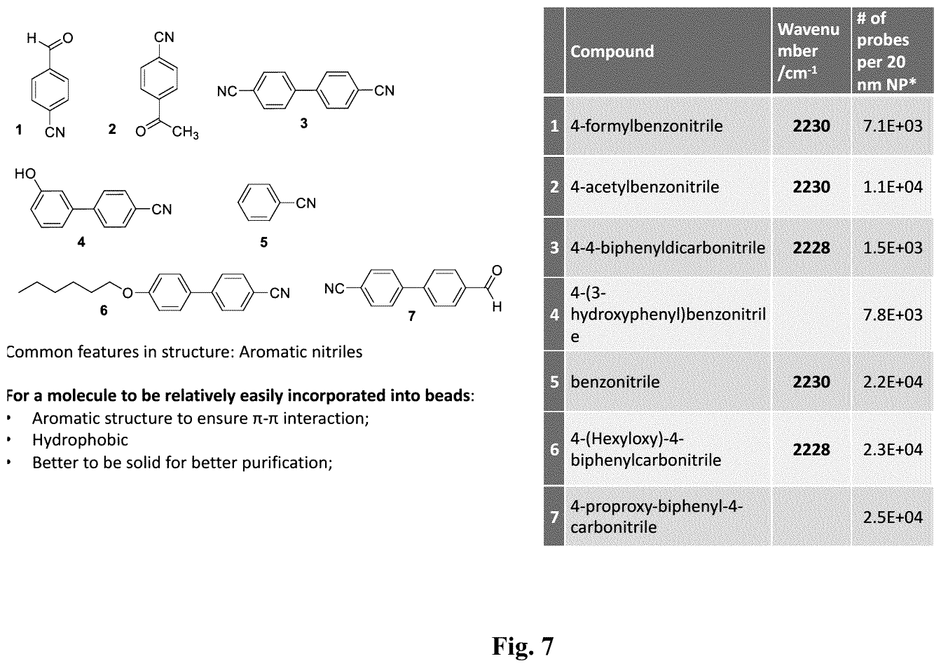

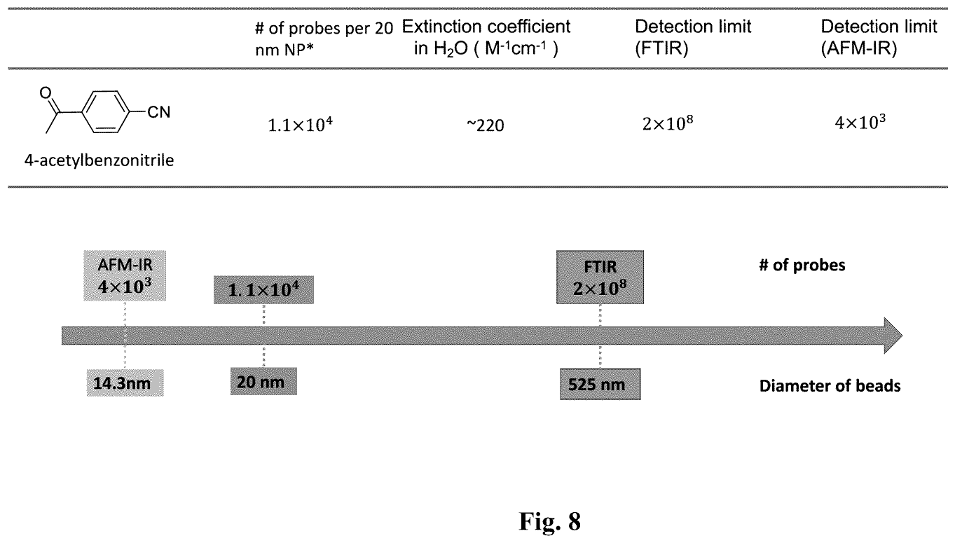

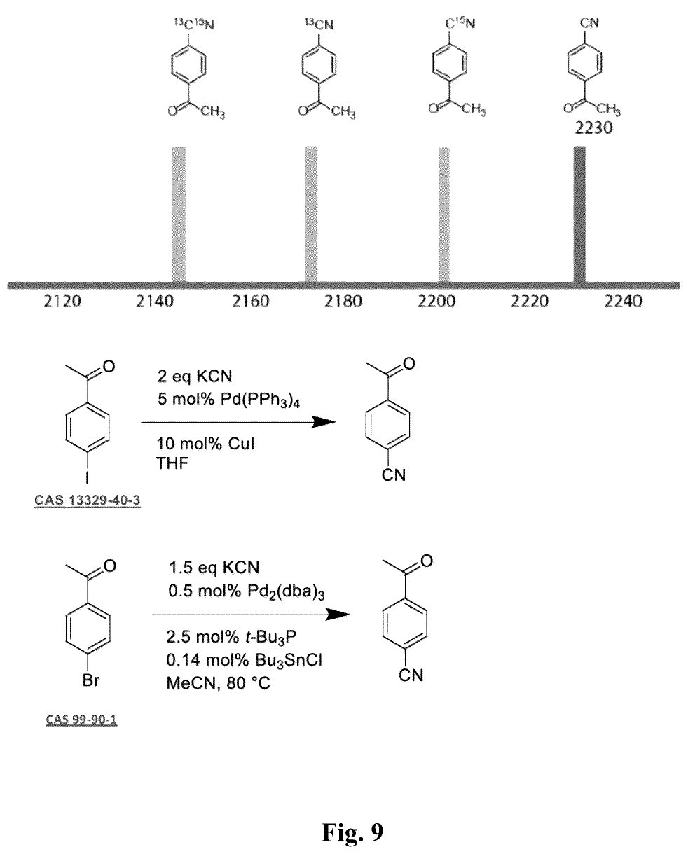

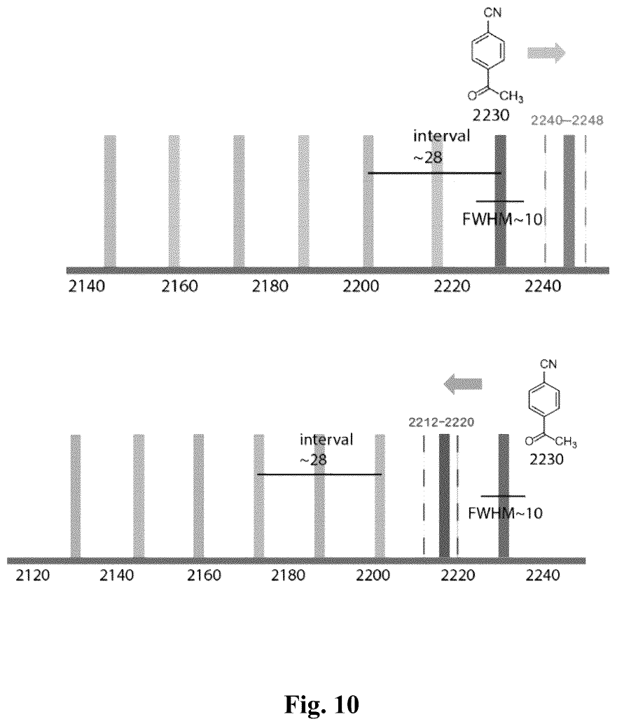

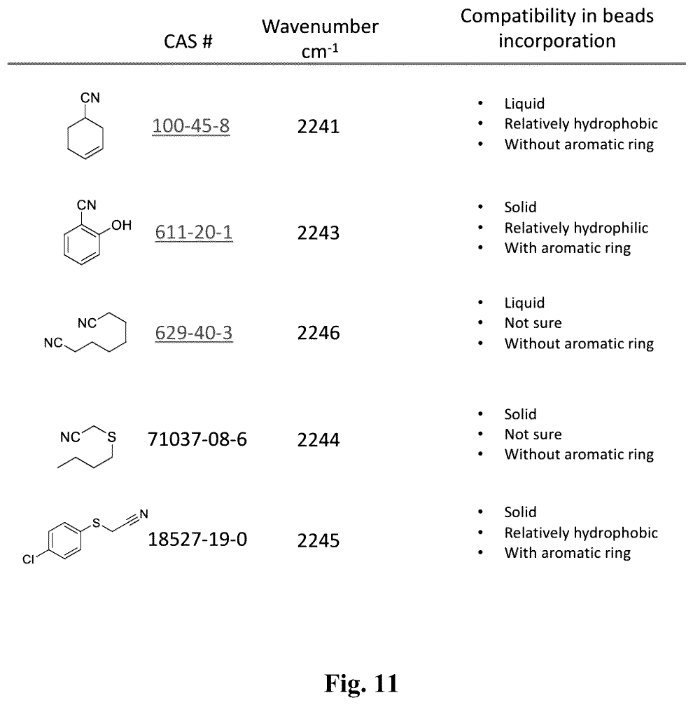

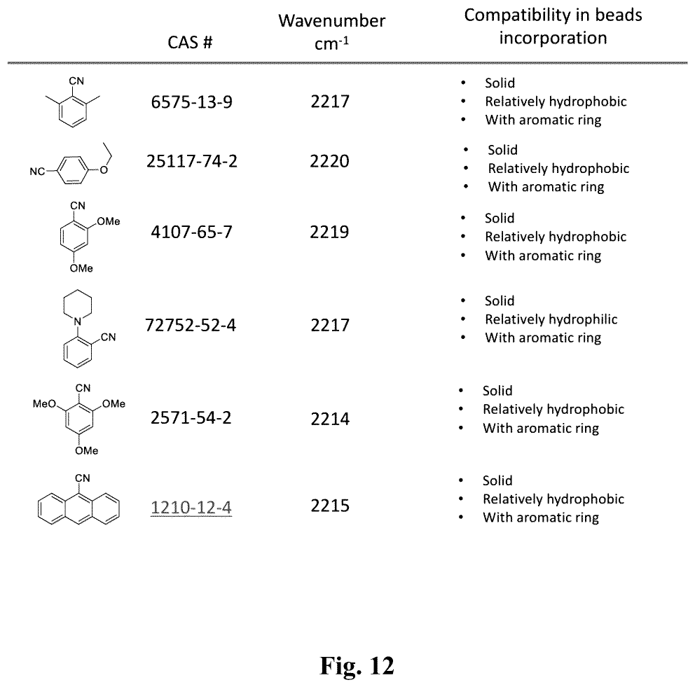

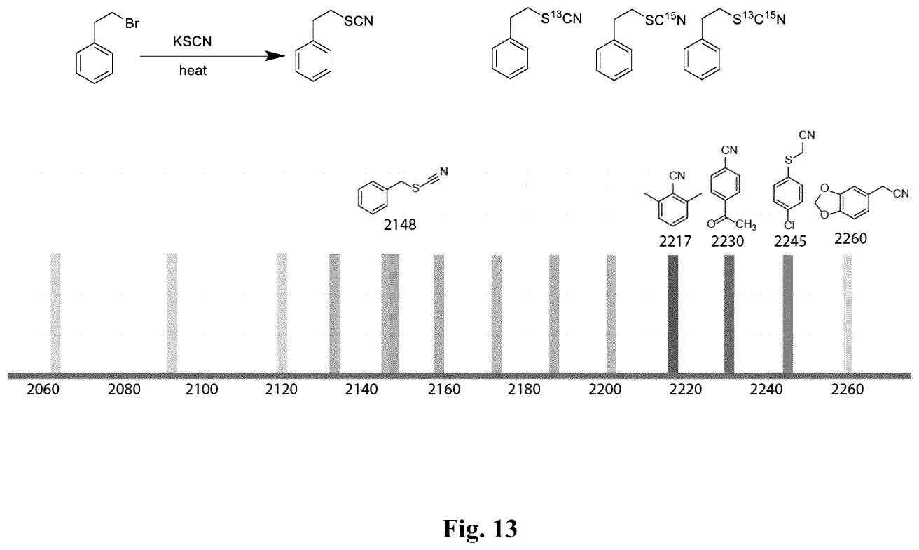

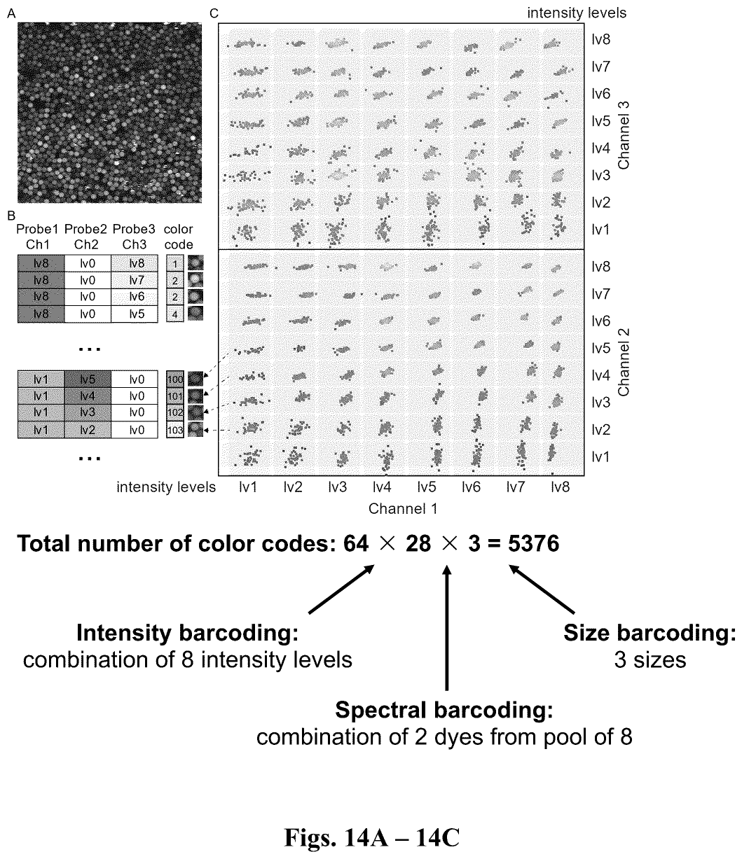

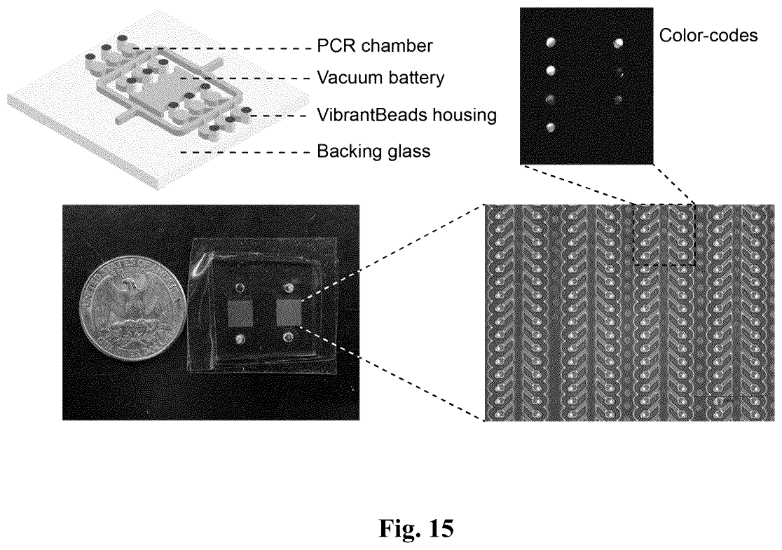

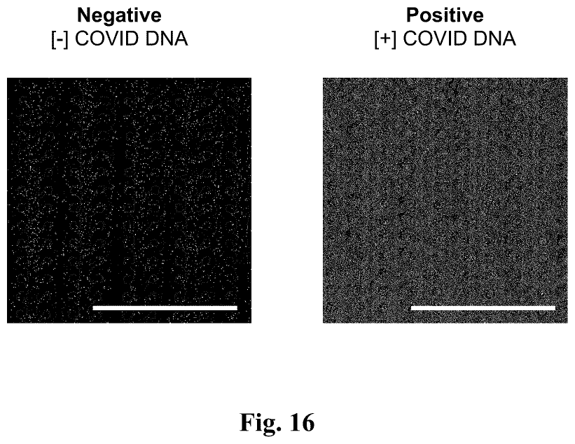

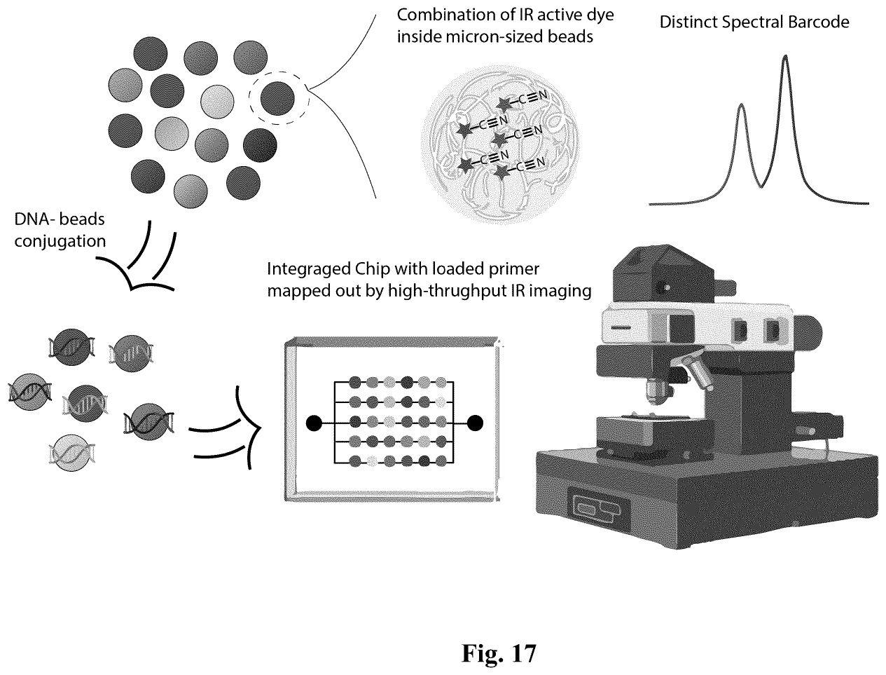

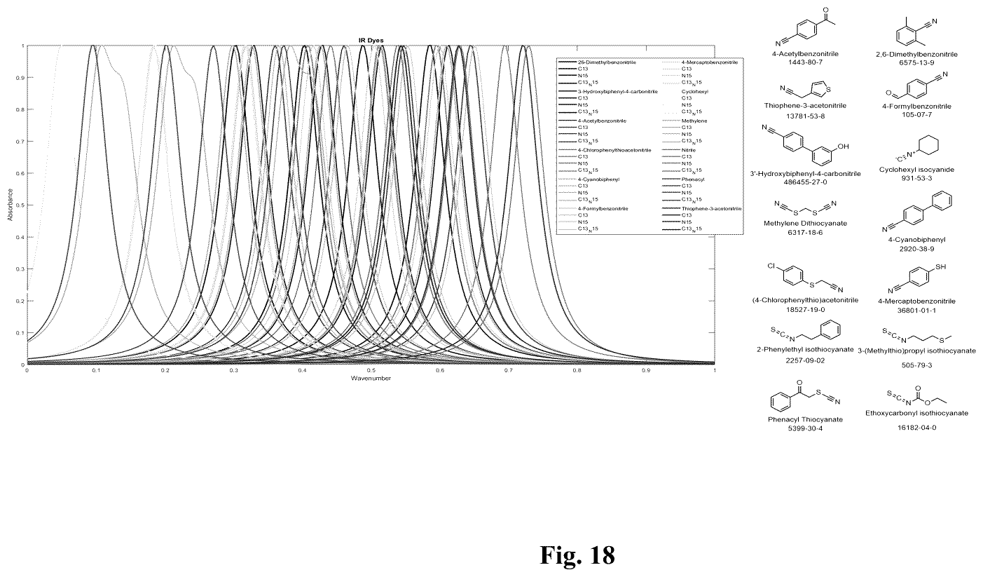

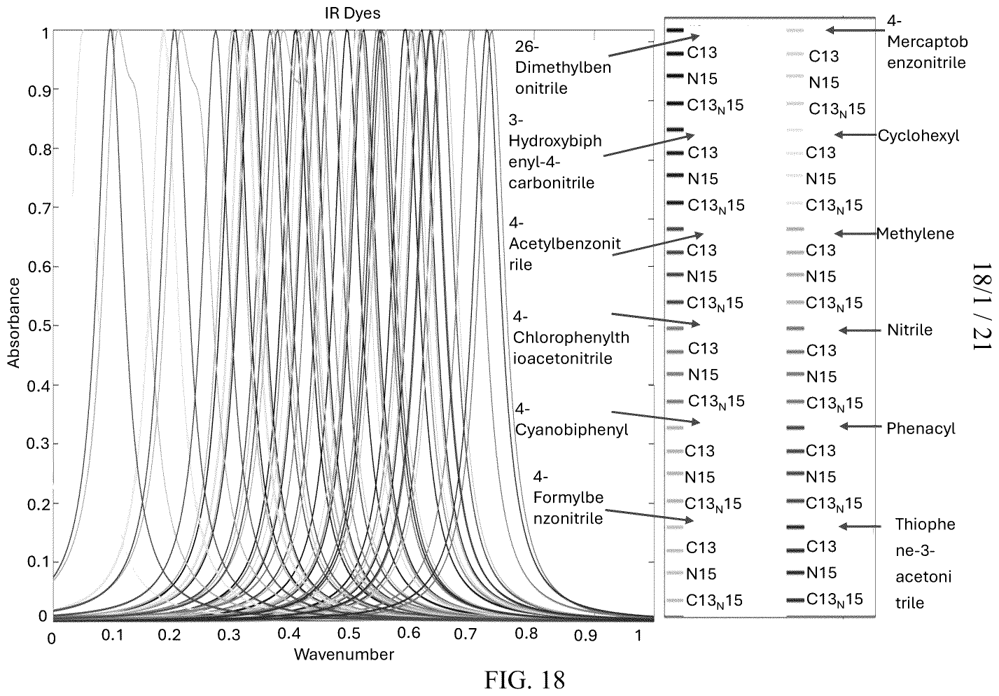

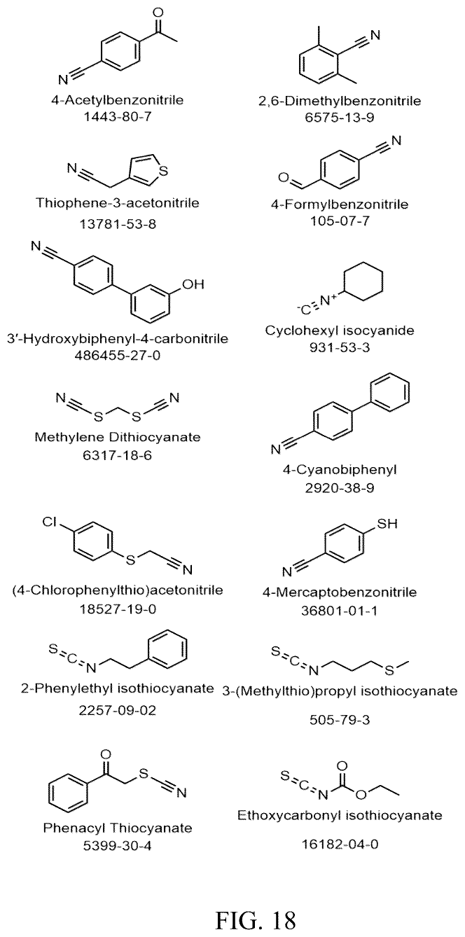

In embodiments, IRASMs may comprise one or more of the molecules shown in FIG. 5 through FIG. 13. In embodiments, IRASMs may comprise one or more of the molecules shown in Attachment A.

In embodiments, the IRAMS are nitrile bond-containing.

In embodiments, the chip comprises at least 1,000 microwells, each microwell with a microbead contained therein, having covalently or non-covalently attached thereto at least one pair of primers comprising a forward and reverse primer specific for a predefined nucleic acid, wherein no microwell contains a pair of primers having the same sequences as a pair of primers in any other microwells of the chip.

In embodiments, the chip comprises at least 10,000 microwells, each microwell with a microbead contained therein, having covalently or non-covalently attached thereto at least one pair of primers comprising a forward and reverse primer specific for a predefined nucleic acid, wherein no microwell contains a pair of primers having the same sequences as a pair of primers in any other microwells of the chip.

In embodiments, the chip comprises at least 100,000 microwells, each microwell with a microbead contained therein, having covalently or non-covalently attached thereto at least one pair of primers comprising a forward and reverse primer specific for a predefined nucleic acid, wherein no microwell contains a pair of primers having the same sequences as a pair of primers in any other microwells of the chip.

In embodiments, the chip comprises at least 500,000 microwells, each microwell with a microbead contained therein, having covalently or non-covalently attached thereto at least one pair of primers comprising a forward and reverse primer specific for a predefined nucleic acid, wherein no microwell contains a pair of primers having the same sequences as a pair of primers in any other microwells of the chip.

In embodiments, the microwell array chip comprises for every known human bacterial pathogen, or a subset thereof, a microwell with a microbead contained therein, having covalently or non-covalently attached thereto at least one pair of primers comprising a forward and reverse primer specific for a nucleic acid distinct to that human bacterial pathogen.

In embodiments, the pathogen is a respiratory pathogen. In embodiments, the pathogen is a blood pathogen. In embodiments, the pathogen is a gastrointestinal pathogen. In embodiments, the pathogen is a CNS pathogen.

In embodiments, the microwell array chip comprises for every known human viral pathogen, or a subset thereof, a microwell with a microbead contained therein, having covalently or non-covalently attached thereto at least one pair of primers comprising a forward and reverse primer specific for a nucleic acid distinct to that human viral pathogen.

In embodiments, the primers are attached to the microbead via a covalent bond.

In embodiments, the primers are attached to the microbead via a poly(U) sequence.

In embodiments, the microwell array chip comprises in each well, a buffer solution, a USER (Uracil-Specific Excision Reagent) enzyme (or an enzyme(s) having DNA glycosylase and a endonuclease VIII enzyme activities), a Taq polymerase (or DNA polymerase suitable for PCR), a mixture of dNTPs for PCR, and a marker which changes its detectable signal upon binding to double-stranded DNA.

In embodiments, the marker which changes its detectable signal upon binding to double-stranded DNA is SYBR® green dye or a BRYT green® dye or EvaGreen® dye. Many such double-stranded DNA binding cyanine dyes are known and encompassed by the invention.

In embodiments, the primers are attached to the microbead via a non-covalent bond.

In embodiments, the primers are attached to the microbead via a heat-labile non-covalent bond.

In embodiments, in each well, a buffer solution, a Taq polymerase, a mixture of dNTPs for PCR, and a marker which changes its detectable signal upon binding to double-stranded DNA. In embodiments, the marker which changes its detectable signal upon binding to double-stranded DNA is SYBR® green dye or a BRYT green® dye or EvaGreen® dye.

In embodiments, the microwell array chip comprises buffer and sample nucleic acid and PCR reagents within microwells of the plurality, and an aqueous liquid-impermeable barrier atop one or more of the microwells preventing flow of the microwell contents of one microwell to any other microwell of the chip.

In embodiments, the aqueous liquid-impermeable barrier comprises an oil.

In embodiments, the oil is a floral oil or a mineral oil.

In embodiments, the each microwell does not comprise a spatially separated signal detection portion in fluid communication with portion of the microwell containing the microbead. In embodiments, the spatially separated signal detection portion does not contain the microbead therein. In embodiments, the spatially separated signal detection portion is too small for microbead to be contained therein.

In embodiments, each microwell is 101% to 195% the diameter of the microbead. In embodiments, each microwell is 101% to 150% the diameter of the microbead. In embodiments, each microwell is 110% to 150% the diameter of the microbead.

In embodiments, each microwell has a volumetric capacity of between 1 aL to about 1 μL, optionally between about 10 aL to about 1 μL or optionally between about 1 fL to about 1 μL. In embodiments, each microwell has a volumetric capacity of between 10 and 100 pL.

In embodiments, all microbead types are of the same size, or are all of about the same size. In embodiments, each microbead type of the plurality has an average diameter of 0.5 μm. In embodiments, each microbead type of the plurality has an average diameter of 1.0 μm. In embodiments, each microbead type of the plurality has an average diameter of 3.0 μm. In embodiments, each microbead type of the plurality has an average diameter of 5.0 μm. In embodiments, each microbead type of the plurality has an average diameter of 10.0 μm. In embodiments, each microbead type of the plurality has an average diameter of not less than 1.0 μm. In embodiments, each microbead type of the plurality has an average diameter of not less than 750 nm. In embodiments, each microbead type of the plurality has an average diameter of no greater than 0.5 μm. In embodiments, each microbead type of the plurality has an average diameter of no greater than 1.0 μm. In embodiments, each microbead type of the plurality has an average diameter of no greater than 1.5 μm.

A microwell chip array is substantially planar, thin device comprising multiple microwells. The microwell chip array can be rigid, semi-rigid, or flexible. The term “thin” refers to a thickness dimension that is 10 mm or less such as between 10 mm and 0.1 mm, and can be about 3 mm, about 2.5 mm, about 2 mm, about 1.5 mm, about 1 mm, or about 0.5 mm.

In embodiments, the microwell chip array has dimensions of 0.1 mm×0.1 mm. In embodiments, the microwell chip array has dimensions of 0.2 mm×0.2 mm. In embodiments, the microwell chip array has dimensions of 0.3 mm×0.3 mm. In embodiments, the microwell chip array has dimensions of 0.4 mm×0.4 mm. In embodiments, the microwell chip array has dimensions of 0.5 mm×0.5 mm. In embodiments, the microwell chip array has dimensions of 1.0 cm×1.0 cm. In embodiments, the microwell chip array has dimensions of 1.5 cm×1.5 cm. In embodiments, the microwell chip array has dimensions of 2.0 cm×2.0 cm. In embodiments, the microwell chip array has dimensions of from 0.5 cm to 1.5 cm×0.5 cm to 1.5 cm. In embodiments, the microwell array chip is circular, and has a diameter of one of the foregoing listed dimensions.

The term “microbeads” refers to solid phase members such as particles, granules or microspheres, typically polystyrene, PMMA, polyacrylamide gel, paramagnetic or magnetic microspheres.

Some or all of the neighboring microwells in an array can have a separation distance of between 1 μm-10 mm, such as about 1 μm, 2 μm, 3 μm, 4μ, 5 μm, 6 μm, 7μ, 8μ, 9 μm, 10 μm, 11 rpm, 12 μm, 13 μm, 14 μm, 15 μm, 16 μm, 17 μm, 18 μm, 19 μm, 20 μm, 100 μm, 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm, 9 mm, 10 mm, or even greater and any fractional number therebetween, measured centerline to centerline of respective neighboring microbead-containing microwell.

In performing the methods of the invention, one can optionally subtract background microbead autofluorescence, but this step is not required. In some embodiments, no measuring or subtraction of microbead background autofluorescence is performed. In this regard, there is no need to spatially or physically separate the bead and its fluorescence signal from a detection region of the MWA chip. In some embodiments, the detection of double-stranded amplicons is performed from the region of the microwell above or below the microbead. In embodiments, the detection of double-stranded amplicons is performed from the region of the microwell aside or adjacent to the region containing the microbead, wherein no microbead is in the line of light travel to and/or from the detector. In general, the prior art has required that a signal detection region spatially distinct from the bead-containing region of a micro array is required for the ability to read the assay signal. For example, a portion of a well spatially and/or physically away from a microbead's background fluorescence makes singleplex reactions in a compact array possible in low volume wells using, e.g., SYBR Green®, has been required in other devices and methods. However, the present invention, in some embodiments, does not require such a spatial distinction, and the singleplex reactions in a compact array are possible in low volume wells using SYBR Green®, or other double-stranded DNA binding dyes that change their signal upon binding double-stranded DNA.

The predefined nucleic acids can be any for which a primer pair sequence can be determined or is known.

In embodiments, the predefined nucleic acids are from human respiratory viral pathogens. For example: Adenovirus, Coronavirus HKU1, Coronavirus NL63, Coronavirus 229E, Coronavirus OC43, Human Metapneumovirus, Human Rhinovirus/Enterovirus, Influenza A, Influenza A/H1, Influenza A/H3, Influenza A/H1-2009, Influenza B, Parainfluenza Virus 1, Parainfluenza Virus 2, Parainfluenza Virus 3, Parainfluenza Virus 4, and Respiratory Syncytial Virus.

In embodiments, the predefined nucleic acids are from human respiratory bacterial pathogens. For example, Bordetella pertussis, Chlamydophila pneumoniae, and Mycoplasma pneumoniae.

In embodiments, the predefined nucleic acids are from human gastrointestinal viral pathogens. For example: Adenovirus F40/41, Astrovirus, Norovirus GI/GII, Rotavirus A, and Sapovirus (I, II, IV, and V).

In embodiments, the predefined nucleic acids are from human gastrointestinal bacterial pathogens. For example: Campylobacter (jejuni, coli and upsaliensis), Clostridium difficile (Toxin A/B), Plesiomonas shigelloides, Salmonella, Yersinia enterocolitica, Vibrio (parahaemolyticus, vulnificus and cholerae), Vibrio cholerae, Diarrheagenic E. coli/Shigella, Enteroaggregative E. coli (EAEC), Enteropathogenic E. coli (EPEC), Enterotoxigenic E. coli (ETEC) It/st, Shiga-like toxin-producing E. coli (STEC) stx 1/stx2, E. coli O157, and Shigella/Enteroinvasive E. coli (EIEC).

In embodiments, the predefined nucleic acids are from human gastrointestinal parasitic pathogens. For example: Cryptosporidium, Cyclospora cayetanensis, Entamoeba histolytica, and Giardia lamblia.

In embodiments, the predefined nucleic acids are from human blood pathogens. For example: pathogenic gram-negative bacteria or pathogenic gram-positive bacteria. For example: Enterococcus, Listeria monocytogenes, Staphylococcus, Streptococcus, Staphylococcus aureus, Streptococcus agalactiae, Streptococcus pneumoniae, and Streptococcus pyogenes. For example: Acinetobacter baumannii, Haemophilus influenzae, Neisseria meningitidis, Pseudomonas aeruginosa, Enterobacteriaceae, Enterobacter cloacae complex, Escherichia coli, Klebsiella oxytoca, Klebsiella pneumoniae, Proteus, and Serratia marcescens. In embodiments, the predefined nucleic acids are from human blood pathogens that are yeast. For example: Candida albicans, Candida glabrata, Candida krusei, Candida parapsilosis, and Candida tropicalis.

In embodiments, the predefined nucleic acids are from CNS pathogens, e.g., as found in CSF. For example, bacterial CNS pathogens such as: Escherichia coli K1, Haemophilus influenzae, Listeria monocytogenes, Neisseria meningitidis, Streptococcus agalactiae, and Streptococcus pneumoniae. For example, viral CNS pathogens such as: Cytomegalovirus (CMV), Enterovirus, Epstein-Barr virus (EBV), Herpes simplex virus 1 (HSV-1), Herpes simplex virus 2 (HSV-2), Human herpesvirus 6 (HHV-6), Human parechovirus, and Varicella zoster virus (VZV). For example, yeast CNS pathogens such as: Cryptococcus gattii, and Cryptococcus neoformans.

The analyzed sample can be any analyte of interest from a sample including, for example, DNA, RNA, and/or various mixtures of DNA and RNA. The sample can be, or be derived from, biofluids, blood, serum, urine, dried blood, cell growth media, lysed cells, beverages or food, and can include environmental samples such as water, air, or soil. Samples include, without limitation, saliva, blood, CSF, mucus, nasal discharge, GI samples/stool samples, plasma, urine, sweat, and swabbed fluids from, e.g., mouth, lung, vagina, colon.

In embodiments, the predefined nucleic acid sequence(s) are from pathogens. In embodiments, the pathogen(s) are human pathogens.

Embodiments of human bacterial pathogens:
Actinomyces israelii
Bacillus anthracis
Bacteroides fragili
Bordetella pertussis
Borrelia
B. burgdorferi
B. garinii
B. afzelii
B. recurrentis
Brucella
B. abortus
B. canis
B. melitensis
B. suis
Campylobacter jejuni
Chlamydia
C. pneumoniae
C. trachomatis
Chlamydophila psittaci
Clostridium
C. botulinum
C. difficile
C. perfringens
C. tetani
Corynebacterium diphtheriae
Ehrlichia
E. canis
E. chaffeensis
Enterococcus
E. faecalis
E. faecium
Escherichia
E. coli (generally)
Enterotoxigenic E. coli (ETEC)
Enteropathogenic E. coli
Enteroinvasive E. coli (EIEC)
Enterohemorrhagic (EHEC), including E. coli 0157: H7
Francisella tularensis
Haemophilus influenzae
Helicobacter pylori
Klebsiella pneumoniae
Legionella pneumophila
Leptospira species
Listeria monocytogenes
Mycobacterium
M. leprae
M. tuberculosis
Mycoplasma pneumoniae
Neisseria
N. gonorrhoeae
N. meningitidis
Pseudomonas aeruginosa
Nocardia asteroides
Rickettsia rickettsii
Salmonella
S typhi
S. typhimurium
Shigella
S. sonnei
S. dysenteriae
Staphylococcus aureus
epidermidis
saprophyticus
Streptococcus agalactiae
pneumoniae
pyogenes
viridans
Treponema pallidum subspecies pallidum
Vibrio cholerae
Yersinia pestis.
Embodiments of human viral pathogens:
Adeno-associated virus
Aichi virus
Australian bat lyssavirus
Banna virus
Barmah forest virus
BK polyomavirus
Bunyamwera virus
Bunyavirus La Crosse
Bunyavirus snowshoe hare
Cercopithecine herpesvirus
Chandipura virus
Chikungunya virus
Cosavirus A
Cowpox virus
Coxsackievirus
Crimean-Congo hemorrhagic fever virus
Dengue virus
Dhori virus
Dugbe virus
Duvenhage virus
Eastern chimpanzee simian foamy virus
Eastern equine encephalitis virus
Ebolavirus
Echovirus
Encephalomyocarditis virus
Epstein-Barr virus
European bat lyssavirus
GB virus C/Hepatitis G virus
Hantaan virus
Hendra virus
Hepatitis A virus
Hepatitis B virus
Hepatitis C virus
Hepatitis delta virus
Hepatitis E virus
Horsepox virus
Human adenovirus
Human astrovirus
Human coronavirus
Human cytomegalovirus
Human enterovirus
Human herpesvirus 1
Human herpesvirus 2
Human herpesvirus 6
Human herpesvirus 7
Human herpesvirus 8
Human immunodeficiency virus
Human papillomavirus 1
Human papillomavirus
Human papillomavirus 2
Human parainfluenza
Human parvovirus B19
Human respiratory syncytial virus
Human rhinovirus
Human SARS coronavirus
Human T-lymphotropic virus
Human torovirus
Influenza A virus
Influenza B virus
Influenza C virus
Isfahan virus
Japanese encephalitis virus
JC polyomavirus
Junin arenavirus
KI Polyomavirus
Lagos bat virus
Lake Victoria marburgvirus
Langat virus
Lassa virus
Louping ill virus
Lymphocytic choriomeningitis virus
Machupo virus
Mammalian orthorubulavirus 5 (Simian virus 5)
Mayaro virus
Measles virus
Merkel cell polyomavirus
MERS coronavirus
Mokola virus
Molluscum contagiosum virus
Monkeypox virus
Mumps virus
Murray valley encephalitis virus
New York virus
Nipah virus
Norwalk virus
O′nyong-nyong virus
Orf virus
Oropouche virus
Pichinde virus
Poliovirus
Punta toro phlebovirus
Puumala virus
Rabies virus
Rift valley fever virus
Rosavirus A
Ross river virus
Rotavirus A
Rotavirus B
Rotavirus C
Rubella virus
Sagiyama virus
Salivirus A
Sandfly fever Naples phlebovirus (Toscana virus)
Sandfly fever sicilian virus
Sapporo virus
SARS coronavirus 2
Semliki forest virus
Seoul virus
Simian foamy virus
Sindbis virus
Southampton virus
St. louis encephalitis virus
Tick-borne powassan virus
Torque teno virus
Uukuniemi virus
Vaccinia virus
Varicella-zoster virus
Variola virus
Venezuelan equine encephalitis virus
Vesicular stomatitis virus
West Nile virus
Western equine encephalitis virus
WU polyomavirus
Yaba monkey tumor virus
Yaba-like disease virus
Yellow fever virus
Zika virus.

Primers for amplification are known in the art and are generally short, single-stranded oligonucleotides (including naturally occurring oligonucleotides such as DNA and synthetic and/or modified oligonucleotides) of any suitable length, but are typically from 5, 6, or 8 nucleotides in length up to 40, 50, or 60 nucleotides in length, or more, and are complementary to a predefined target sequence. In embodiments, polymerase chain reaction (PCR) is employed and may be carried out in accordance with known techniques and methods in the art. In general, PCR involves, first, treating a nucleic acid sample (e.g., in the presence of a heat stable DNA polymerase) with a first oligonucleotide primer which hybridizes to a strand of a specific target sequence and a second oligonucleotide primer which hybridizes to a complementary strand of the specific target sequence under hybridizing conditions. An extension product of each primer is synthesized under extension conditions, and thus each extension product formed by one primer is complementary to each other extension product formed by the other primer through the corresponding region where the other primer hybridized its specific target nucleic acid strand. The primers have sufficient complementary to each strand of the specific target sequence (i.e., the target the sequence strand and its complementary strand) to hybridize therewith so that the extension product synthesized from each primer, when it is separated from its complement, can serve as a template for synthesis of the extension product of the other primer. Then treating the sample under denaturing conditions to separate the primer extension products from their templates. Cycles of hybridization (or “annealing), extension, and denaturation are typically repeated a sufficient number of times to exponentially generate amplicon molecules, with each strand of the amplicon extending to the binding site of the corresponding primer. Accordingly, if the sequence or sequences to be detected are present in a sample, PCR may be used to amplify such sequences. In embodiments, where isothermal amplification is employed, the number of primers depends on the technique. For example, Loop-mediated Isothermal Amplification (LAMP) can require four (4) or six (6) primers. Strand Displacement Amplification (SDA) can require four (4) primers. Other isothermal amplification techniques require only a pair of primers. In addition, the polymerase employed can be a strand displacement polymerase.

“The term “specific” with respect to a pair of primers (e.g., a forward primer and a reverse primer) refers to a primer pair which is designed for use in amplifying a predefined nucleic acid sequence in a sample during a DNA synthesis reaction (e.g. PCR) without significantly amplifying sequences other than predefined nucleic acid sequence in the sample. For example, a primer pair specific for a predefined nucleic acid sequence is typically used in a PCR reaction to generate amplicons having the predefined sequence without significantly generating amplicons having sequences other than the predefined sequence.

PCR steps are typically cyclically repeated until the desired degree of amplification is obtained.

Any suitable PCR technique may be used, e.g., quantitative PCR (qPCR), reverse transcription PCR (RT-PCR), quantitative reverse transcription PCR (qRT-PCR), etc.

Additional guidance on typical PCR and related techniques may be found in the following brief reviews: Polymerase Chain Reaction (PCR)—www.neb.com/en-us/applications/dna-amplification-pcr-and-qpcr/polymerase-chain-reaction-pcr; Loop-mediated Isothermal Amplification (LAMP)-www.neb.com/en-us/applications/dna-amplification-pcr-and-qpcr/isothermal-amplification/loop-mediated-isothermal-amplification-lamp; www.neb-online.de/en/pcr-and-dna-amplification/isothermal-amplification/; Strand Displacement Amplification (SDA)—www.neb.com/en-us/applications/dna-amplification-pcr-and-qpcr/isothermal-amplification/strand-displacement-amplification-and-nicking-enzyme-amplification-reaction; qPCR and RT-PCR-www.neb.com/en/applications/dna-amplification-pcr-and-qpcr/qpcr-and-rt-qpcr; www.neb.com/en/products/rna-reagents/reverse-transcriptases-and-rt-pcr/reverse-transcriptases-and-rt-pcr; www.neb.com/en/products/pcr-qpcr-and-amplification-technologies/cdna-synthesis-and-reverse-transcriptases/cdna-synthesis-and-reverse-transcriptases.

In embodiments, DNA amplification techniques such as the foregoing can involve the use of a pair of primers which specifically bind to DNA containing a polymorphism or mutation of interest, but do not bind to DNA that does not contain the polymorphism of interest under the same hybridization conditions, and which serve as the primers for the amplification of the DNA or a portion thereof in the amplification reaction.

In embodiments, the method is completed within 30 minutes of placing sample within the microwell. In embodiments, the method is completed within 60 minutes of placing sample within the microwell. In embodiments, the method is completed within 1200 minutes of placing sample within the microwell.

The term “reagent” refers to any substance or compound, including primers, the nucleic acid template and the amplification enzyme, that is added to a system in order to bring about a chemical reaction, or added to see if a reaction occurs. In an embodiment, the reagents are amplification reagents. Amplification reagents or reagent refer to those reagents (deoxyribonucleotide triphosphates, buffer, etc.) generally used for amplification except for primers, nucleic acid template and the amplification enzyme.

The term “marker” refers to a molecule which displace a detectable signal under certain conditions. For example, a marker molecule may emit a signal (e.g., a fluorescence signal) when in the presence of certain types of molecules (e.g., double-stranded DNA).

In embodiments, a system is provided comprising (i) a detector component which can detect a light signal from a marker within one or more microwells of a microwell array chip, which marker changes its detectable signal upon binding to double-stranded DNA, and (ii) a microwell array chip as described herein.

In embodiments, the marker which changes its detectable signal upon binding to double-stranded DNA is a green light-emitting or cyanine-based dye. In embodiments, the marker is a SYBR dye, SYBR® green dye, a BRYT green® dye, or an EvaGreen® dye.

In embodiments, the SYBR dye is SYBR-1 (N′,N′-dimethyl-N-[4-[(E)-(3-methyl-1,3-benzothiazol-2-ylidene)methyl]-1-phenylquinolin-1-ium-2-yl]-N-propylpropane-1,3-diamine).

In embodiments, the system further comprises an instrument which sequentially subjects the sample in each microwell of the microwell array chip to nucleic acid amplification conditions and then melting conditions, wherein the instrument is configured to provide a first number of amplification cycles, then perform a first melt, and output an amplicon detection and/or quantification result for each microwell.

In embodiments, the instrument is programmed to tune the number of amplification cycles to the titers of the predefined nucleic acids.

In embodiments, the detector component is part of an image-processing device which has a resolution that spatially resolves the marker signal from each microwell of the microwell array chip, thereby individually detecting by signal presence and/or quantitating by signal strength the amplicons for each microwell.

In embodiments, the detector component comprises a camera.

In embodiments, the system comprises a computer processor which is programmed with the identity of the predefined nucleic acid that each microwell of the microwell array chip contains a primer pair specific for, thereby permitting identification of the nucleic acid whose amplicon is detected and/or quantified in one or more microwell(s) of the microwell array chip, thus identifying which predefined nucleic acid(s) are present in the sample and/or in what amount.

In embodiments, the system comprises a computer processor which is programmed with the identity of the predefined nucleic acid at each microwell location on the microwell array chip for which the microbead contained therein comprises a primer pair specific for, thereby permitting detection of the presence or not of the predefined nucleic acid whose amplicon is present by detection of a signal indicating the presence of a double-stranded DNA therein.

In embodiments, the system is a portable system and/or handheld system.

Also provided is a method is provided for determining the presence of one or more predefined nucleic acids in a sample, the method comprising adding the sample to a microwell of (a) the microwell array (MWA) chip as described herein or (b) the microwell array (MWA) chip of the system as described herein, wherein the MWA chip contains a microwell for each predefined nucleic acid having a microbead therein with a pair of primers comprising a forward and reverse primer specific for said predefined nucleic acid, and cleaving the covalent or non-covalent bond between each primer and the microbead and having therein provided, or adding, reagents sufficient to permit a polymerase chain reaction to occur, so as to produce one or more double-stranded amplicons of the predefined nucleic acid(s), and detecting the presence of the double-stranded amplicons for the one or more predefined nucleic acids, wherein presence of the double stranded amplicons indicates that the predefined nucleic acid is/are present in the biological sample.

Also provided is a method for determining the presence of one or more predefined nucleic acids in a sample, the method comprising adding the sample to (a) a microwell of a microwell array (MWA) chip described herein or (b) to a microwell of a microwell array (MWA) chip of a system described herein, wherein the MWA chip contains a microwell for each predefined nucleic acid having a microbead therein with at least one pair of primers covalently or non-covalently attached thereto comprising a forward and reverse primer specific for said predefined nucleic acid, and cleaving the covalent or non-covalent bond between each primer and the microbead and having therein provided, or adding, reagents sufficient to permit an isothermal amplification reaction to occur therein, so as to produce one or more double-stranded amplicons of the predefined nucleic acid(s), and detecting the presence of the double-stranded amplicons, wherein presence of the double stranded amplicons indicates that the predefined nucleic acid is/are present in the biological sample.

In embodiments of the methods, the method also comprises determining the quantity of double-stranded amplicons.

In embodiments of the methods, the microbeads are treated so as to cleave off the primers attached thereto when or after the sample is added to the microwell.

In embodiments of the methods, the primers are each covalently attached to their microbeads by a poly(U) sequence and wherein cleavage thereof is effected by contacting with a USER (Uracil-Specific Excision Reagent) enzyme.

In embodiments of the methods, the primers are non-covalently attached to the microbead by heat-labile bond and cleavage thereof is effected by heating.

In embodiments of the methods involving PCR, wherein the primers are non-specifically absorbed by the microbeads, they are able to detach at PCR conditions of high temp, relatively high salt. In embodiments, wherein the primers are incorporated in polyacrylamide gel, e.g., via monomers with di-sulfo bonds, the gel can be dissolved to release the primers with the presence of reducers. Reducers include DTT, which is commonly found in PCR buffer.

In embodiments of the methods involving PCR, reagents sufficient to permit a polymerase chain reaction to occur can comprise a buffer solution, a Taq polymerase, a mixture of dNTPs.

In embodiments of the methods involving isothermal amplification, reagents sufficient to permit an isothermal amplification reaction to occur can comprise a buffer solution, a polymerase such as EquiPhi29 or Bsm DNA polymerase or Bst DNA polymerase, and/or a mixture of dNTPs. In embodiments, wherein Loop-Mediated Isothermal Amplification (LAMP) is employed, reagents can include 4-6 different primers (e.g., two or three pairs of specific primers in each microwell) and strand displacing enzyme(s). In embodiments, the methods employ an isothermal amplification technique. In embodiments, the technique is one of Nucleic Acid Sequence-based Amplification (NASBA), Loop-mediated Isothermal Amplification (LAMP), Strand Displacement Amplification (SDA), Recombinase Polymerase Amplification (RPA) or Rolling Circle Amplification (RCA). In embodiments, the technique involves no temperature cycling. In embodiments, the methods employ one pair of specific primers per microbead. In embodiments, the methods employ two different pairs of specific primers per microbead. In embodiments, the methods employ three different pairs of specific primers per microbead.

In embodiments of the methods, detecting the presence of the double-stranded amplicons is effected by detecting a marker which changes its detectable signal upon binding to double-stranded DNA.

In embodiments, the marker which changes its detectable signal upon binding to double-stranded DNA is SYBR® green dye or a BRYT green® dye or EvaGreen® dye.

In embodiments, the methods further comprise, after adding the sample to a microwells of the MWA chip, adding an aqueous liquid-impermeable barrier atop the microwell(s) preventing flow of microwell contents to any other microwell of the chip.

In embodiments, the aqueous liquid-impermeable barrier comprises an oil.

In embodiments, the oil is a floral oil or a mineral oil.

In embodiments, the method determines the presence of more than one predefined nucleic acid simultaneously in a biological sample, and the method comprises distributing the sample to a first plurality of microwells of the microwell array chip, wherein in each microwell of said first plurality the pair of primers comprising a forward and reverse primer are specific for a predefined nucleic acid, and wherein each microwell of the first plurality contains (i) a pair of primers having different sequences from other pairs of primers in microwells of said microwell array chip, and/or (ii) a pair of primers having specific for a predefined nucleic acid for which no other microwell of the first plurality contains a pair of primers having specific for.

In embodiments, the microwell array chip also comprises a second plurality of microwells, wherein each microwell of said second plurality contains, attached to a microbead therein, a pair of primers specific for a predefined nucleic acid for which a microwell of the first plurality also contains a pair of primers specific for.

Also provided is a multi-well plate comprising a plurality of wells, wherein each well is not in fluid contact with any adjacent well and has a volume of between 0.5 milliliters and 5 milliliters, and wherein each well further comprises, on a bottom surface thereof, a microwell array chip as described herein.

Also provided is a method of manufacturing a microwell array chip comprising
printing a microwell array design on a soda lime photomask;
spin-coating a photoresist layer onto a silicon wafer;
exposing through the photomask, developing and hard-curing the photoresist layer on the silicon wafer so as to produce a microwell array-patterned silicon wafer;
vacuum-depositing trichloro(1H, 1H,2H,2H-perfluorooctyl) silane onto the silicon wafer;
pouring uncured and degassed PDMS polymer onto the patterned silicon wafer;
and a) heat curing the PDMS polymer so as to form a PDMS microwell array chip; or b) placing a thin coverglass on top so that the PDMS polymer is sandwiched between the patterned silicon wafer and coverglass and applying pressure and heat to spread the PDMS across the coverglass substantially evenly and pushing the patterned silicon wafer to touch the coverglass, while applying heat for rapid curing so as to form a glass-bottomed PDMS microwell array chip; and removing the PDMS or glass-bottomed PDMS microwell array chip from the patterned silicon wafer; and
adding to each microwell of the microwell array chip a predetermined microbead comprising one, or more, types of Raman-active small molecule(s) (RASMs), each at a predefined concentration in the microbead, or one or more, types of infrared-active (IR) small molecule(s) (IRASMs), each at a predefined concentration in the microbead,
thereby producing a microwell array chip.

In embodiments, each microbead is brightness and spectra barcoded as described herein.

In embodiments, the mask printer has sufficient resolution to produce the microwell pattern of the dimensions described herein.

In embodiments, the method of manufacture further comprises, prior to or subsequently to adding to each microwell of the microwell array a predetermined microbead, attaching to the microbead covalently or non-covalently, at least one pair of primers comprising a forward and reverse primer specific for a predefined nucleic acid.

In embodiments, heat curing is performed for 15 min set at 150° C. In embodiments, a heat press is employed to spread PDMS.

In embodiments, the method of manufacture further comprises making the PDMS polymer fresh before use by mixing SYLGARD 184 and a curing reagent at 10:1 weight ratio.

In embodiments, the PDMS is degassed under vacuum degassing. In embodiments, the degassed under vacuum degassing is performed for 30 min.

In embodiments, the method of manufacture further comprises functionalizing surfaces of the microwells of the microwell array chip with amine groups. In embodiments, functionalizing surfaces of the microwells of the microwell array chip with amine groups improves hydrophilicity and interaction between well walls and microspheres. In embodiments, functionalizing surfaces of the microwells of the microwell array chip with amine groups comprises contacting the surfaces with 3-aminopropyltriethoxysilane (APTES) in a suitable solvent, incubating, drying, and washing. In embodiments, the solvent is ethanol solution (1:2 v/v). In embodiments, the washing is effected with aqueous acetic acid or ammonia incubation. In embodiments, the aqueous acetic acid is 33% w/v aqueous acetic acid.

A method is provided for determining the presence of one or more predefined nucleic acids in a sample, the method comprising adding the sample to a microwell of the (a) microwell array (MWA) chip as described herein or (b) microwell array (MWA) chip of the system as described herein, wherein the MWA chip contains a microwell for each predefined nucleic acid having a microbead therein with a pair of primers comprising a forward and reverse primer specific for said predefined nucleic acid, and cleaving the covalent or non-covalent bond between each primer and the microbead and having therein provided, or adding, reagents sufficient to permit a polymerase chain reaction to occur or reagents sufficient to permit an isothermal amplification reaction to occur therein, so as to produce one or more double-stranded amplicons of the predefined nucleic acid(s), and isolating the double-stranded amplicons from each microwell and performing a sequencing technique thereupon.

In embodiments, the sequencing technique is a next generation sequencing technique.

In embodiments, the method optionally comprises, prior to isolating the double-stranded amplicons from each microwell, detecting the presence of the double-stranded amplicons for the one or more predefined nucleic acids.

A method is provided for determining the presence of one or more predefined nucleic acids in a sample, the method comprising adding the sample to a microwell of the (a) microwell array (MWA) chip comprising a plurality of microwells,
wherein each microwell of the plurality has a microbead contained therein which has, covalently or non-covalently attached thereto, at least one pair of primers comprising a forward and reverse primer specific for a predefined nucleic acid,
and wherein the microbead of each microwell is individually resolvable from that of every other microbead in the plurality of microwells having a different at least one pair of primers comprising a forward and reverse primer specific for a predefined nucleic acid, wherein the MWA chip contains a microwell for each predefined nucleic acid having a microbead therein with a pair of primers comprising a forward and reverse primer specific for said predefined nucleic acid,
   and cleaving the covalent or non-covalent bond between each primer and the microbead and having therein provided, or adding, reagents sufficient to permit a polymerase chain reaction to occur therein, or reagents sufficient to permit an isothermal amplification reaction to occur therein, so as to produce one or more double-stranded amplicons of the predefined nucleic acid(s).

In embodiments, the method optionally comprises further performing targeted sequencing upon said amplicons. In embodiments, the microbead of each microwell is individually resolvable from that of every other microbead in the plurality of microwells based on its spatial coordinates within the MWA.

An embodiment of a PCR manufacturing overview:
1. Microspheres are doped with a combination of various dyes to optically barcode by both brightness and spectra.
2. Each kind of barcode-resolvable microsphere is surface functionalized with a pair of primers.
3. Optically barcoded and primer conjugated microspheres are loaded to a microwell array (MWA) chip and dried.
4. Optical barcodes of each microsphere in the MWA are read so that the identities of primers are known in each microwell.
5. Samples and reagents mixture for PCR are introduced to the MWA chip. The reagent mixture contains enzymes needed to cleave the primers from microspheres. MWA chip is then sealed.
6. Chips are then placed on a customized PCR machine with proper imaging system to image the fluorescence signal from each microwell, in real-time. qPCR reaction happens individually in each microwell according to the identity of the microspheres. Since there could be 100-1,000,000 different kinds of microspheres, as many as 1 million qPCR can occur simultaneously.
7. Data analysis is carried out to correlate the fluorescence signal curve to the identity of each microwell.

An embodiment of an isothermal amplification manufacturing overview:
1. Microspheres are doped with a combination of various dyes to optically barcode by both brightness and spectra.
2. Each kind of barcode-resolvable microsphere is surface functionalized with a pair of primers, or more than one different pair depending on which isothermal amplification method is employed.
3. Optically barcoded and primer conjugated microspheres are loaded to a microwell array (MWA) chip and dried.
4. Optical barcodes of each microsphere in the MWA are read so that the identities of primers are known in each microwell.
5. Samples and reagents mixture for isothermal amplification are introduced to the MWA chip. The reagent mixture contains enzymes needed to cleave the primers from microspheres. MWA chip is then sealed.
6. Chips are then placed on a customized isothermal amplification machine (which does not require a thermal cycler) with proper imaging system to image the fluorescence signal from each microwell, in real-time. An isothermal amplification reaction happens individually in each microwell according to the identity of the microspheres. Since there could be 100-1,000,000 different kinds of microspheres, as many as 1 million isothermal amplifications can occur simultaneously.
7. Data analysis is carried out to correlate the fluorescence signal curve to the identity of each microwell.

### Optical Barcoded Microspheres:

We have previously proposed and demonstrated how to optically barcode microspheres with Raman-active-small-molecules (RASMs) to generate “Vibrant MicroBeads”. These barcodes will then be readout with Raman spectroscopy. We now extend the approach to using infrared (IR) imaging. In this approach, a variety of IR-active-small-molecules (IRASMs) are effectively doped into microspheres to generate Vibrant MicroBeads, using the same brightness and spectra barcoding technique.

This strategy was achieved by two dimensions of optical barcoding: brightness barcoding and spectral barcoding. Assuming: 1) each one of the IRASM can be doped into microbeads at M different concentrations, resulting in microbeads that have M brightness levels (brightness barcoding); 2) a combination of n IRASMs can be doped together into one microbead and the Raman signal of each IRASM in the same microbead can be detected individually (spectral barcoding), then the total possible number of unique Vibrant MicroBeads would be Mn⁻¹. If 5 brightness barcoding and 10 spectral barcoding were considered, the resulting unique Vibrant MicroBeads would be 5¹⁰⁻¹=9,765,624 flavors. This massive multiplexity is the direct result of the fact that these IRASMs (like RASMs) have individually resolvable IR spectra, whereas, for most fluorescence-based techniques, only about three (3) spectrally resolvable fluorescence dyes can be used at the same time, greatly limiting the multiplexity.

In addition to the brightness barcoding and spectral barcoding mechanism, size barcoding can also be used where various sizes of microbeads can be used together to further increase the multiplexity. These size barcoding can either be read out by taking images and directly measuring the diameter of each microbead or by quantitatively measuring the forward and backward scattering as the scattering strength is dependent on their sizes. When size barcoding is made use of, the total multiplexity would be S*(Mⁿ⁻¹) where S indicates the number of different sizes.

### Primer Conjugation to Microspheres:

Since the primers for each individual PCR reaction (or isothermal amplification reaction) in the microwell are ‘delivered’ by Vibrant MicroBeads, the attachment of these primers to their corresponding Vibrant MicroBeads is important. This can be achieved both by covalently linked to and/or by noncovalently attaching the primers to the microbeads.

### Covalent Linkage:

Amine-modified primers can be efficiently linked to carboxyl modified microbeads through aminecarboxylic acid coupling. In this setting, the primers can include consecutive deoxy-uridine on the 5′ end, so that when a USER enzyme is added to the PCR mixture, the primers will be cleaved off the microbead surface and thus used for PCR reactions. Alternatively, the USER enzyme can also be used for the technique involving isothermal amplification, permitting the cleaved off primers to engage in isothermal amplification reactions.

### Non-Covalent Linkage:

Examples include heat-labile bonds. Other examples include surfaces of the microbeads modified with streptavidin, and thus allowing biotin to bind the modified microbeads. The primers can be absorbed nonspecifically to the microbeads with proper buffer (similar to the way that magnetic beads are used for DNA isolation). A shell of polyacrylamide gel with primers in it can be formed around the microbeads. Upon gel polymerization, the primers will remain in the shell of gel around microbeads.

### Microwell Array Chips:

A high density microwell array was designed and fabricated with photolithography techniques. The design principle was simply to have microwells that can only allow one Vibrant MicroBead to settle in.

The following procedures can be followed to fabricate the microwell arrays:
1. MWA design is printed on a soda lime photomask with suitable mask printer with enough resolution.
2. A layer of ˜20 μm thick SU8 photoresist is spin-coated onto a silicon wafer, following the recommended protocols from the manufacturer.
3. Photoresist coated wafer is then exposed, developed, and hard cured per manufacturer's recommendation.
4. A layer of trichloro(1H, 1H,2H,2H-perfluorooctyl) silane is then vacuum deposited onto the wafer to facilitate PDMS release.
5. PDMS polymer is made fresh by mixing SYLGARD 186 and the curing reagent at 10:1 weight ratio. The mixture is then under vacuum degassing for 30 min.
6. Two approaches are developed to fabricate PDMS chips and glass-bottomed PDMS chips. For PDMS chip, uncured and degassed PDMS is directly poured onto the patterned silicon wafer. After another 30 min vacuum degassing, the PDMS is then allowed to heat cure according to the manufacturer's manual. After PDMS curing, the PDMS chip can be peeled off from the wafer. For glass-bottomed PDMS chips, uncured and degassed PDMS is poured onto the patterned silicon wafer, then a piece of thin cover glass is placed on top so that the PDMS is sandwiched by the wafer and glass. Pressure and heat is applied by a heat press for 15 min set at 150° C. to spread the PDMS across the glass evenly and push the SU8 patterns to touch the glass, while providing the heat necessary for rapid curing. After PDMS curing, the glass-bottomed PDMS chip can be peeled off from the wafer.
7. The chip is then surface functionalized with amine groups to improve hydrophilicity and interaction between well walls and microspheres. 3-aminopropyltriethoxysilane (APTES):ethanol solution (1:2 v/v) is added to the chip and incubate for 5 min, and then dried, followed by 33% w/v aqueous acetic acid or ammonia incubation for 5 min. The chip is then washed with DI water and dried. Finally the RASM or IRASM barcoded microbeads are added—one microbead to each microwell (with or without primer pairs attached thereto).

One unexpected property we found with the MWA chip disclosed herein is that once the Vibrant MicroBeads were settled in the microwells, they stay in the microwells even with vigorous pipetting or drying. This may be due to the attraction between the positively charged microwells from the amine modification and negative charges. This unique feature allows us to pre-fabricate MWA chip with the described microbeads in the microwells thereof. The chip can then be dried and characterized with either a Raman spectrometer or an IR spectrometer to read the individual microbead identity for each microwell thereof.

In embodiments, the types of microbead are further barcoded by size difference. In embodiments, size barcoding is read by taking images and measuring the diameter of each microbead. In embodiments, size barcoding is read quantitatively by measuring the forward and backward scattering (since the scattering strength is dependent on their sizes).

In embodiments, the plurality of microbeads (with one microbead of the plurality per microwell) comprises 5 or more different brightness levels. In embodiments, the microbeads comprise 10 or more different brightness levels. In embodiments, the microbeads comprise 15 or more different brightness levels.

In embodiments, the plurality of microbeads (with one microbead of the plurality per microwell) comprises at least 90 different bead types. In embodiments, the microbeads comprise at least 100 different bead types. In embodiments, the microbeads comprise at least 500 different bead types. In embodiments, the microbeads comprise at least 1000 different microbead types. In embodiments, the microbeads comprise at least 10,000 different microbead types. In embodiments, the microbeads comprise at least 100,000 different microbead types. In embodiments, the microbeads comprise at least 500,000 different microbead types. In any of the above embodiments, the microbeads comprise up to 1,000,000 different microbead types, or more.

See doi.org/10.1038/s41467-021-21570-0 for doping a large number of Raman-active-small-molecules (RASMs) efficiently into polymer particles via non-covalent interactions.

Isotopically-labeled compounds can generally be prepared by conventional techniques known to those skilled in the art using appropriate isotopically-labeled reagents in place of the non-labeled reagents employed.

The term “alkynyl” herein refers to a hydrocarbon radical straight or branched, containing at least 1 carbon-to-carbon triple bond—an “alkyne”—and up to the maximum possible number of non-aromatic carbon-carbon triple bonds may be present. Thus, C2-Cn alkynyl is defined to include groups having 1, 2 . . . , n−1 or n carbons. For example, “C2-C6 alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C2-Cn alkynyl. An embodiment can be C2-C12 alkynyl or C3-C8 alkynyl.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

For the foregoing embodiments, each embodiment disclosed herein is contemplated as being applicable to each of the other disclosed embodiment.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1. A microwell array (MWA) chip comprising a plurality of microwells,
wherein each microwell contains a microbead that is covalently or non-covalently attached to at least one pair of primers comprising a forward primer and reverse primer, wherein the pair of primers is specific for a predefined nucleic acid, and wherein
(a) each microbead comprises
one or more types of Raman-active small molecule(s) (RASMs), each at a predefined concentration in the microbead, or
one or more types of infrared-active (IR) small molecule(s) (IRASMs), each at a predefined concentration in the microbead; and
(b) the Raman spectrum or IR spectrum of each microbead containing the one or more RASMs or IRASMs is individually resolvable from that of every other microbead in the plurality of microwells attached to a different at least one pair of primers.

2. The microwell array chip of claim 1, wherein the plurality of microwells comprises 50-100, 100-500, 500-1,000, 1,000-10,0000, 10,000-50,000, 50,000-100,000, 100,000-500,000, 500,000-1,000,000, or more microwells.

3. The microwell array chip of claim 1, wherein the plurality of microwells comprises 500,000 or more microwells.

4. The microwell array chip of claim 1, wherein each microwell of the plurality is sized such that only one microbead can be contained therein.

5. The microwell array chip of claim 1, wherein each microwell of the plurality comprises a polydimethylsiloxane wall or walls and a polydimethylsiloxane bottom, or comprises a polydimethylsiloxane wall or walls and a glass bottom.

6. The microwell array chip of claim 1, wherein surfaces of the microwell, which surfaces are in contact with a fluid sample when the fluid sample is placed in the microwell, are functionalized with amine groups.

7. The microwell array chip of claim 6, wherein the amine-functionalized surfaces increase hydrophilicity so as to increase interaction between well walls and microspheres as compared to a non-functionalized surface of the microwell.

8. The microwell array chip of claim 1, wherein each microbead comprises a polystyrene particle.

9. The microwell array chip of claim 1, wherein each microbead has a diameter of about 0.5-1.0μ, 1.0-3.0 μm, 3.0-5.0 μm, or 5.0-10.0 μm.

10. The microwell array chip of claim 1, wherein each microbead has peak Raman shift at a predetermined stimulation wavelength of at least 10 cm⁻¹ less or 10 cm⁻¹ more than the peak Raman shift of all the other microbead types.

11. The microwell array chip of claim 1, wherein the RASMs are alkyne-containing and do not exceed a molecular weight of 350 g/mol.

12. The microwell array chip of claim 1, wherein the IRASMs are nitrile bond-containing.

13. The microwell array chip of claim 1, wherein the chip comprises at least 1,000, preferably 1,000-10,000, or more, microwells, each microwell containing a microbead that is covalently or non-covalently attached to at least one pair of primers comprising a forward primer and a reverse primer specific for a predefined nucleic acid, wherein no microwell contains a pair of primers having the same forward primer and reverse primer sequences as a pair of primers in any other microwell of the chip.

14. The microwell array chip of claim 1, wherein the chip comprises at least 10,000, preferably 10,000-100,000, or more, microwells, each microwell containing a microbead that is covalently or non-covalently attached to at least one pair of primers comprising a forward primer and a reverse primer specific for a predefined nucleic acid, wherein no microwell contains a pair of primers having the same forward primer and reverse primer sequences as a pair of primers in any other microwell of the chip.

15. A system comprising (i) a detector component which can detect a light signal emitted from a marker within one or more microwells of a microwell array chip, which marker changes its detectable signal upon binding to double-stranded DNA, and (ii) the microwell array claim 1.

16. A method for determining the presence of one or more predefined nucleic acids in a sample, the method comprising adding the sample to a microwell of the (a) microwell array (MWA) claim 1, wherein the MWA chip contains a microwell for each predefined nucleic acid having a microbead therein with a pair of primers comprising a forward primer and a reverse primer specific for said predefined nucleic acid, and cleaving the covalent or non-covalent bond between each primer and the microbead and having therein provided, or adding, reagents sufficient to permit a polymerase chain reaction to occur or reagents sufficient to permit an isothermal amplification reaction to occur therein, so as to produce one or more double-stranded amplicons of the predefined nucleic acid(s), and detecting the presence of the double-stranded amplicons for the one or more predefined nucleic acids, wherein presence of the double stranded amplicons indicates that the predefined nucleic acid is/are present in the biological sample.

17. A multi-well plate comprising a plurality of wells, wherein each well is not in fluid contact with any adjacent well and has a volume of between 0.5 milliliters and 5 milliliters, and wherein each well further comprises, on a bottom surface thereof, a microwell array chip of claim 1.

18. A method of manufacturing a microwell array chip comprising
printing a microwell array design on a soda lime photomask;
spin-coating a photoresist layer onto a silicon wafer;
exposing through the photomask, developing and hard-curing the photoresist layer on the silicon wafer so as to produce a microwell array-patterned silicon wafer;
vacuum-depositing trichloro(1H, 1H,2H,2H-perfluorooctyl)silane onto the silicon wafer;
pouring uncured and degassed PDMS polymer onto the patterned silicon wafer;
and a) heat curing the PDMS polymer so as to form a PDMS microwell array chip; or b) placing a thin coverglass on top so that the PDMS polymer is sandwiched between the patterned silicon wafer and coverglass and applying pressure and heat to spread the PDMS across the coverglass substantially evenly and pushing the patterned silicon wafer to touch the coverglass, while heat for rapid curing so as to form a glass-bottomed PDMS microwell array chip; and
removing the PDMS or glass-bottomed PDMS microwell array chip from the patterned silicon wafer; and
adding to each microwell of the microwell array a predetermined microbead comprising one, or more, types of Raman-active small molecule(s) (RASMs), each at a predefined concentration in the microbead, or one or more, types of infrared-active (IR) small molecule(s) (IRASMs), each at a predefined concentration in the microbead, thereby producing a microwell array chip.

19. A method for determining the presence of and/or quantifying one or more predefined nucleic acids in a sample, the method comprising adding the sample to a microwell of the (a) microwell array (MWA) chip of claim 1, wherein the MWA chip contains a microwell for each predefined nucleic acid having a microbead therein with a pair of primers comprising a forward primer and reverse primer specific for said predefined nucleic acid, and cleaving the covalent or non-covalent bond between each primer and the microbead and having therein provided, or adding, reagents sufficient to permit a polymerase chain reaction to occur or reagents sufficient to permit an isothermal amplification reaction to occur therein, so as to produce one or more double-stranded amplicons of the predefined nucleic acid(s), and isolating double-stranded amplicons from each microwell and performing a sequencing technique thereupon.

20. A method for determining the presence of and/or quantifying one or more predefined nucleic acids in a sample, the method comprising adding the sample to a microwell of a microwell array (MWA) chip comprising a plurality of microwells,
wherein each microwell of the plurality has a microbead contained therein which has, covalently or non-covalently attached thereto, at least one pair of primers comprising a forward primer and a reverse primer specific for a predefined nucleic acid,
and wherein the microbead of each microwell is individually resolvable from that of every other microbead in the plurality of microwells having a different at least one pair of primers,
wherein the MWA chip contains a microwell for each predefined nucleic acid,
and cleaving the covalent or non-covalent bond between each primer and the microbead and having therein provided, or adding, reagents sufficient to permit a polymerase chain reaction to occur therein, or reagents sufficient to permit an isothermal amplification reaction to occur therein, so as to produce one or more double-stranded amplicons of the predefined nucleic acid(s).
